(19) United States
(12) Patent Application Publication
(10) Pub. No.: US 2012/0309697 A1
(43) Pub. Date: Dec. 6, 2012

(54) METHODS OF DIAGNOSING AND PROGNOSING COLONIC POLYPS

(76) Inventors: Samuel Norbert Breit, Gordon (AU); David Alexander Brown, Lane Cove (AU); Kenneth W. Hance, Groton, MA (US); Elaine Lanza, Clearwater, FL (US); Connie J. Rogers, Olney, MD (US)

(21) Appl. No.: 13/504,154

(22) PCT Filed: Oct. 27, 2010
(86) PCT No.: PCT/AU2010/001432

§ 371 Date: Jul. 9, 2012

(30) Foreign Application Priority Data
Oct. 28, 2009 (AU) 2009905277

(51) Int. Cl. G01N 33/566; A61P 1/00; A61P 9/00; A61P 35/00; A61P 39/00; A61P 13/12; A61P 3/04; A61P 21/00; A61P 3/00; A61P 15/00; A61K 38/19; A61P 9/10

(52) U.S. Cl. 514/21.92; 435/7.92; 435/7.1; 436/501

(57) ABSTRACT

Methods of diagnosing or prognosing a disease or condition associated with increased or over expression of macrophage inhibitory cytokine-1 (MIC-1) are disclosed. The methods typically involve detecting a change in the amount of MIC-1 in a test body sample from a subject taken at two or more timing points. The change in the amount of MIC-1 may be adjusted for the effect of at least the following factors as appropriate: the gender of the subject, the age of the subject, the body mass index (BMI) of the subject, the subject being a smoker, the subject being a user of NSAIDs, and the waist-to-hip ratio where the subject is female. The methods are particularly suitable for diagnosing or prognosing the presence of one or more colorectal polyp(s).

### FIELD OF THE INVENTION

The present invention relates to, methods of diagnosing or prognosing a disease or condition associated with increased or over expression of macrophage inhibitory cytokine-1 (MIC-1). In a particular application, the invention relates to a method of diagnosing or prognosing the presence of one or more colorectal polyp(s) in a subject.

### INCORPORATION BY REFERENCE

This patent application claims priority from:
Australian Provisional Patent Application No 2009905277 titled “Methods of diagnosing and prognosing colonic polyps” filed 28 Oct. 2010.

The entire content of this application is hereby incorporated by reference.

The following patent specifications are referred to herein:
International Patent Specification No WO 01/81928 titled “Diagnostic assay and method of treatment involving macrophage inhibitory cytokine (MIC-1)”, and
International Patent Specification No WO 2009/052557 titled “Methods of prognosis”.

The entire content of these specifications is also hereby incorporated by reference.

### BACKGROUND TO THE INVENTION

MICA is a transforming growth factor-β (TGF-β) superfamily protein. MIC-1 was originally cloned as macrophage inhibitory cytokine-1 and later identified as placental transforming growth factor-β (PTGF-β), placental bone morphogenetic protein (PLAB), non-steroidal anti-inflammatory drug-activated gene 1 (NAG-1), prostate-derived factor (PDF) and growth development factor-15 (GDF-15) (Bootcov et al., 1997; Hromas et al., 1997; Lawton et al., 1997; Yokoyama-Kobayashi et al., 1997; Paralkar et al., 1998). Similar to other TGF β-related cytokines, MIC-1 is synthesised as an inactive precursor protein, which undergoes disulphide-linked dimerisation. Upon proteolytic cleavage of the N-terminal pro-peptide, mature MIC-1 is secreted as an approximately 24.5 kDa dimeric protein (Bauskin et al., 2000). Amino acid sequences for MIC-1 are disclosed in WO 99/06445, WO 00/70051, WO 01/8.1928, WO 2005/113585, Bottler et al. (1999b), Bootcov et al., 1997, Baek et al., 2001, Hromas et al. (1997), Paralkar et al., 1998, Morrish et al., 1996, and Yokoyama-Kobayashi et al., (1997). The amino acid sequence for the common or “wild type” mature human MIC-1 polypeptide monomer is shown at FIG. 1.

MIC-1 can be expressed in several tissues (Moore et al., 2000; Bottner et al., 1999a; Fairlie et al., 1999; Bauskin et al., 2006). For example, Northern blots of human tissues indicate the presence of small amounts of MIC-1 mRNA in the kidney, pancreas and prostate, and large amounts in the placenta (Moore et al., 2000; Fairlie et al., 1999). Further, serum MIC-1 levels have been shown to increase with age in normal, apparently healthy subjects (Brown et al., 2006). In addition, serum MIC-1 is elevated in chronic inflammatory diseases (Brown et al., 2007) and predicts atherosclerotic events independently of traditional risk factors (Brown et al., 2002). Moreover, serum MIC-1 levels are also increased in chronic kidney disease (CKD; Johen et al., 2007), and MIC-1 over expression has been associated with cancer (Welsh et al., 2003). Indeed, serum levels of MIC-1 can serve as a biomarker of cancer as elevated serum MIC-1 has been reported in subjects with a cancer of the prostate (Brown et al., 2006; Selander et al., 2007; Welsh et al., 2601; Welsh et al., 2003), pancreas (Koopman et al., 2004; Koopman et al., 2006), breast (Welsh et al., 2003) and colorectum (Brown et al., 2003). Also, in a case-control study, MIC-1 expression in colon cancer was confirmed by immunohistochemistry and it was observed that there was a progressive increase in serum MIC-1 concentrations (mean±SD) from healthy subjects (495±210 pg/ml) to subjects with colonic polyps (681±410 pg/ml) to subjects with colonic carcinomas (783±491 pg/ml) (Brown et al., 2003). Further, high-grade dysplastic polyps, which have a propensity to progress to malignant colonic carcinoma show higher serum MIC-1 levels, compared with subjects having other types of polyps. Also, in colonic carcinoma, serum MIC-1 concentrations rise further with advancing TNM stage and metastatic disease. As such, high serum MIC-1 levels, prior to treatment of colonic carcinoma, indicate a higher risk of earlier relapse of disease and significantly worse overall survival (Brown et al., 2003).

The present applicants have now found that increased MIC-1 expression is associated with the presence of colorectal polyps and, further, that the changes in MIC-1 expression over time are more strongly associated with the presence or recurrence of colorectal polyps after their initial diagnosis and removal.

Colorectal polyps, otherwise known as colonic polyps and colonic adenomas, are abnormal but non-cancerous tissue growths occurring in the epithelium of the colon. While colorectal polyps are usually symptom-free (nb. they can cause occasional rectal bleeding and, rarely, pain, diarrhoea, torsion, obstruction, intussuception or constipation), the presence of such polyps can be of considerable concern due to their propensity to transform from a benign growth into a malignant colorectal cancer. Most colorectal cancers arise from colonic polyps; hence, detecting and removing colonic polyps greatly reduces the risk of colorectal cancer development.

While investigating the association between MIC-1 expression and colorectal polyps, the present applicants have recognised that the utility of the measurement of MIC-1 levels as the basis for diagnostic and/or prognostic methods for a variety of diseases and conditions can be improved by accounting for: (i) the fact that MIC-1 is present in the blood of all subjects over a broad normal range of about 450-1150 pg/ml such that it is possible for an individual's particular MIC-1 level to more than double and still be within the normal range (nb. MIC-1 is not an acute phase reactant protein and the blood levels of MIC-1 typically remain stable over time; unpublished observations)); and (ii) the fact that other factors (ie other than a disease or condition) can modulate serum MIC-1 levels. Further, the present applicants also investigated whether the use of serial measurements of MIC-1 to provide a measurement of the change in MIC-1 level over time, would provide improved results (ie in the diagnosis and/or prognosis of diseases such as colorectal polyps) over that from a single absolute MIC-1 value itself.

These investigations involved the assessment of MIC-1 levels in blood from a cohort of subjects being evaluated for colorectal polyps in The Polyp Prevention Trial (PPT) (Schatzkin et al., 1996; Lanza et al., 1996; Lanza et at, 2001; Schatzkin et al., 2000), a multicenter randomised clinical trial that allowed for serial measurements of serum MIC-1 concentrations (years 1 and 4), ascertainment of incident colorectal polyps (confirmed by complete colonoscopy with data on the location, multiplicity, and histology of colorectal polyps in year 0), and repeated measurements of dietary intake and the use of prescription and non-prescription medications such as non-steroidal anti-inflammatory drugs (NSAIDs).

### SUMMARY OF THE INVENTION

In a first aspect, the present invention provides a method of diagnosing or prognosing the presence of one or more colorectal polyps in a subject, the method comprising detecting:

(i) a change in the amount of macrophage inhibitory cytokine-1 (MIC-1); and/or
(ii) a shift in rate of change in the amount of MIC-1;
in a test body sample from said subject taken at two or more time points.

Preferably, the change in the amount of MIC-1 or shift in rate of change in the amount of MIC-1 is adjusted for the effect of at least the following factors as appropriate: the gender of the subject, the age of the subject, the body mass index (BMI) of the subject, the subject being a smoker, the subject being a user of NSAIDs, and the waist-to-hip ratio where the subject is female.

A change in the amount of MIC-1, or a shift (namely, an increase) in the rate of change in the amount of MIC-1, may be associated with the presence of one or more colorectal polyp(s) in the subject. Where such a change or shift is detected, the method may further comprise:

(ii) treating said subject so as to remove, reduce or manage the colorectal polyp(s) that are present.

In a second aspect, the present invention provides a method of diagnosing or prognosing a disease or condition in a subject that is associated with increased or over expression of macrophage inhibitory cytokine-1 (MIC-1), the method comprising detecting:

(i) an elevated amount of MIC-1 in a test body sample from said subject, wherein the elevated amount of MIC-1 is associated with said disease or condition;
(ii) a change in the amount of MIC-1 in a test body sample from said subject taken at two or more time points, wherein the elevated change in the amount of MIC-1 is associated with said disease or condition; and/or
(iii) a shift in rate of change in the amount of MIC-1 in a test body sample from said subject taken at two or more time points, wherein the shift in rate of change in the amount of MIC-1 is associated with said disease or condition;
wherein said elevated amount, change in the amount of MIC-1 or shift in rate of change in the amount of MIC-1, is adjusted for the effect of at least the following factors as appropriate: the gender of the subject, the age of the subject, the body mass index (BMI) of the subject, the subject being a smoker, the subject being a user of non-steroidal anti-inflammatory drugs (NSAIDs), and the waist-to-hip ratio where the subject is female.

Where an elevated amount, change in the amount (eg an elevated change) of MIC-1 or a shift (namely, an increase) in the rate of change in the amount of MIC-1, is detected that is associated with the disease or condition in the subject, the method of the third aspect may further comprise:

(iii) treating said subject for said disease or condition.

In a third aspect, the present invention provides a method of prognosis of overall survival of an apparently healthy subject, the method comprising detecting:

(i) an elevated amount of macrophage inhibitory cytokine-1(MIC-1) in a test body sample from said subject, wherein the elevated amount of MIC-1 is associated with an increased likelihood of death of the subject;
(ii) a change in the amount of MIC-1 in a test body sample from said subject taken at two or more time points; and/or
(iii) a shift in rate of change in the amount of MIC-1 in a test body sample from said subject taken at two or more time points;
wherein said elevated amount, change in the amount of MIC-1 or shift in rate of change in the amount of MIC-1, is adjusted for the effect of at least the following factors as appropriate: the gender of the subject, the age of the subject, the body mass index (BMI) of the subject, and the subject being a smoker.

A change in the amount of MIC-1 or a shift in rate of change in the amount of MIC-1, may be associated with an increased likelihood of death of the subject.

### BRIEF DESCRIPTION OF THE FIGURES

FIG. 1 provides (A) the amino acid sequence for the common or “wild type” mature human MIC-1 polypeptide; and (B) the amino acid sequence of a D6 mature human MIC-1 variant.

### DETAILED DESCRIPTION OF THE INVENTION

MIC-1 is commonly overexpressed by many cancers to the extent that this is accompanied by a rise in serum levels of MIC-1. For example, it has been previously reported that elevated levels of serum MIC-1 predict a worse prognosis for colorectal cancer (Brown et al., 2003). In the same study, it was also found that serum MIC-1 levels were elevated in subjects with colorectal polyps, however serum MIC-1 levels remained mostly within the normal range making it difficult to use these data, on their own, as a reliable predictive diagnostic test for colorectal polyps. It has now been found that by using a modified approach, serum MIC-1 levels can indeed be used to reliably predict the presence of colorectal polyps. Further, it has now been found that serum MIC-1 levels can be used to predict polyp recurrence following the removal of colorectal polyps (eg by polypectomy). Indeed, in a particular analysis of data from the above-mentioned PPT, it has been found that multivariate analysis of serial measurements of serum MIC-1 concentrations (years 1 and 4) indicates that the relative risk of polyp recurrence increases with follow-up MIC-1 levels and subjects in the highest quartile of between 1159-6520 pg/mL (P<0.0001) and 40-fold (OR 37.2 95% CI 7.1-195; P<0.0001), depending on the exact analytical approach that is adopted.

Thus, in a first aspect, the present invention provides a method of diagnosing or prognosing the presence of one or more colorectal polyps in a subject, the method comprising detecting:

(i) a change in the amount of macrophage inhibitory cytokine-1 (MIC-1); and/or
(ii) a shift in rate of change in the amount of MIC-1;
in a test body sample from said subject taken at two or more time points.

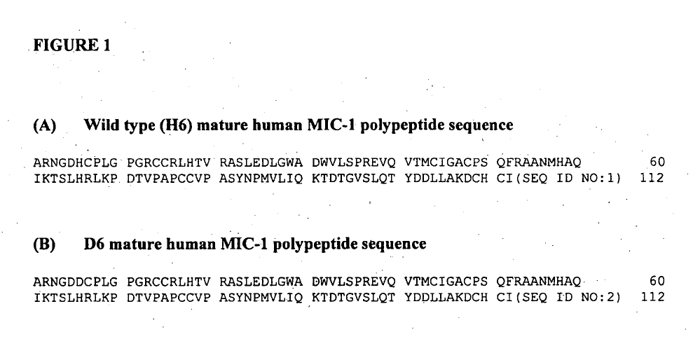

As used herein, the term “MIC-1” encompasses monomers, homodimers and/or heterodimers of a MIC-1 polypeptide, as well as variants, subunits and fragments (eg degradation products or digestion products of MIC-1) thereof. MIC-1 variants encompassed by the term include mature human MIC-1 proteins which comprise a polypeptide comprising an amino acid sequence differing from that shown at FIG. 1A by 1 to 3 amino acids due to an amino acid substitution, deletion and/or addition, and which preferably show substantially equivalent biological activity to the polypeptide comprising the amino acid sequence shown at FIG. 1A (as may be measured using the assay described by Hromas et al., 1997 and/or the test described by Kempf et al., 2007); one particular example is the D6 mature human MIC-1 variant described in WO 01/81928 (the entire content of which is herein incorporated by reference), which comprises the amino acid sequence shown at FIG. 1B. MIC-1 subunits and fragments encompassed by the term include subunits and fragments of the polypeptide comprising the amino acid sequence shown at FIG. 1A or FIG. 1B and which show substantially equivalent immunological and/or biological activity to those polypeptides.

Preferably, the MIC-1 detected in the method of the first aspect is mature human MIC-1 protein, the D6 mature human MIC-1 variant protein and/or heterodimers thereof.

The term “test body sample” as used herein refers to a sample of a body fluid, separated cells (ie cells taken from the body and at least partially separated from other body components), a tissue or an organ. Samples of body fluids can be obtained by well known techniques, and tissue or organ samples may be obtained from any tissue or organ by, for example, biopsy. Separated cells may be obtained from a body fluid, tissue or organ by separating techniques such as centrifugation or cell sorting. Preferably, cell, tissue or organ samples are obtained from those cells, tissues or organs which express or produce MIC-1.

The test body sample for use in the method of the first aspect may, therefore, be preferably selected from whole blood, blood plasma, serum, buffy coat, urine, cerebrospinal fluid, seminal fluid, synovial fluid, a tissue biopsy and/or an organ biopsy. More preferably, the test body sample is selected from the group consisting of whole blood, blood plasma, serum and urine. Most preferably, the test body sample is serum.

The term “amount” as used herein encompasses an absolute amount of MIC-1, a relative amount or concentration of MIC-1 as well as any value or parameter which correlates or corresponds thereto, or can be derived therefrom, such as, for example, values or parameters comprising intensity signal values from all specific physical or chemical properties obtained from MIC-1 by direct measurements (eg intensity values in mass spectra or NMR spectra) or indirect measurements (eg response levels determined from biological read out systems in response to MIC-1 or intensity signals obtained from specifically bound ligands). It is to be understood that values correlating to the abovementioned amounts or parameters can also be obtained by standard mathematical operations well known to persons skilled in the art.

The “change in the amount of MIC-1” for the purposes of the present invention may be represented by an increase, lack of increase or decrease in the amount of MIC-1 within a subject that is detectable by serial measurements. For example, an elevated change in the amount of MIC-1 may be detected by comparing the amount of MIC-1 in a test body sample at a given time point with the amount of MIC-1 in the same test body sample taken at an earlier time point. In this manner, an elevated change in the amount of MIC-1 can be detected by determining an increase in the amount of MIC-1 present in the test body sample within any given subject over time.

The “shift in rate of change in the amount of MIC-1” for the purposes of the present invention may be represented by an increase, lack of increase or decrease in the rate that of change in the amount of MIC-1 within a subject that is detectable by serial measurements. For example, a shift in rate of change in the amount of MIC-1 may be detected by comparing the change in the amount of MIC-1 in a test body sample between, for example, a first and second time point with the rate of change in the amount of MIC-1 between said first and a third time point (ie wherein said third point is subsequent to the second time point) or, preferably, between said second and a third time point. In this manner, a shift in rate of change in the amount of MIC-1 can be detected by determining the rate of change in the amount of MIC-1 present in the test body sample within any given subject over time.

Since MIC-1 levels can be affected by a number of factors (as further described below) including age, it may be anticipated, particularly where the interval between time points is considerable (eg more than 6 months), that the MIC-1 amount will increase or decrease for reasons other than the presence of colorectal polyps. Accordingly, the present applicants have now realised that the detection of a change in the amount of MIC-1, or a shift in rate of change in the amount of MIC-1, in a body sample such as serum between two or more time points, when adjusted for other factors that alter MIC-1 expression or detection, may provide the basis for a more accurate diagnostic or prognostic method. Such other factors include, for example, the particular body sample type used, the gender of the subject (nb. male subjects show a mean serum MIC-1 level higher than that of female subjects), the age of the subject at the initial time point, the body mass index (BMI) of the subject, the smoking status of the subject (nb. former smokers tend to show increased levels of serum MIC-1 compared to those who have never smoked, while current smokers tend to show even greater levels of serum MIC-1), any use of NSAIDs (nb. NSAID use can be associated with increased serum MIC-1 levels, particularly in male subjects, and subsequent cessation may lead to relatively lower serum MIC-1 levels), the waist-to-hip ratio in female subjects, the number and/or size of colorectal polyps present in the subject, when the test body samples were taken, the method used to detect the amounts of MIC-1 and, possibly, the racial origin of the subject, the time of day that the test body samples were taken, and the nature of any exercise undertaken and/or meals consumed by the subject prior to the taking of the test body sample(s).

Preferably, the change in the amount of MIC-1, or shift in rate of change in the amount of MIC-1, is therefore adjusted for the effect of at least the following factors as appropriate: the gender of the subject, the age of the subject, the body mass index (BMI) of the subject, the subject being a smoker, the subject being a user of NSAIDs, and the waist-to-hip ratio where the subject is female. Such adjustment may be made by using any of the multivariate regression analysis techniques well known to persons skilled in the art to produce a weighted score validated for diagnosing or prognosing the presence of one or more colorectal polyp in a subject.

Thus, for example, in the case of a non-smoking male test subject of 45 years of age having a healthy known BMI and; at the time of taking the test body sample, was on a course of NSAIDs, the change in amount referred to in (i) is adjusted for the effect of his gender, age and NSAID use on MIC-1 expression. A particular algorithm based upon multivariate regression that is suitable for adjusting such an elevated change in amount is based upon beta coefficients from Table A including serum MIC-1 quartiles.

TABLE A

| Logistic regression | | | | Number of obs = 317 | | |
|---|---|---|---|---|---|---|
| | | | | L [symbol] chi2(11) = 51.92 | | |
| | | | | Prob > chi2 = 0.0000 | | |
| Log likelihood = −158.34166 | | | | Pseudo R2 = 0.1409 | | |
| recur4 | Coef. | Std. Err. | x | p > \|x\| | [95% Conf. Interval] | |
| mic_1_pg_m-1 | −.001 [symbol] 499 | .0006571 | −2. [symbol] 2 | 0.00 [symbol] | −.003137 [symbol] | −.0005619 |
| 1.drugs_d-14 | −.796 [symbol] | .30 [symbol] 2337 | −2.58 | 0.010 | −1.400412 | −.1921579 |
| age0 | −.01812 [symbol] | .018719 [symbol] | −0.97 | 0.333 | −.05 [symbol] 137 | .01 [symbol] 5645 |
| cic_b [symbol] i_t4 | −.015133 [symbol] | .036909 [symbol] | −0.41 | 0.682 | −.0 [symbol] 7 [symbol] | .05720 [symbol] 5 |
| log10mic-4_q | | | | | | |
| 1 | 1.09 [symbol] 661 | .49711 [symbol] 2 | 2. [symbol] 0 | 0.028 | .1183349 | 2.0669 [symbol] 7 |
| 2 | [symbol] .470666 | .5 [symbol] 61297 | [symbol] .22 | 0.000 | 1.321 [symbol] 73 | 3.619 [symbol] 59 |
| 3 | [symbol] .590479 | . [symbol] 540991 | [symbol] .20 | 0.000 | 1.916476 | 5.26 [symbol] 3 |
| 1. [symbol] x | 1.255 [symbol] 1 | .3090651 | 4.06 | 0.000 | .6500534 | 1.861566 |
| deltamic1 | −.001 [symbol] 1 | .0006 [symbol] 1 | −1. [symbol] 9 | 0.059 | −.002 [symbol] 88 [symbol] | .0000 [symbol] |
| sm_smokcatg | | | | | | |
| 1 | −.461 [symbol] 12 | .31 [symbol] 95 | −1. [symbol] 7 | 0.141 | −1.075179 | .152697 |
| 2 | −.305 [symbol] 31 | . [symbol] 741 [symbol] | −0.68 | 0.49 [symbol] | −1.1 [symbol] 2 [symbol] | .5710231 |
| _co [symbol] | .2471662 | 1.443 [symbol] | 0.17 | 0. [symbol] 6 [symbol] | −2.582637 | 3.07697 |

recur4 = is presence of polyp at time 4 (yes/no);
mic_1_pg_ml-1 = MIC-1 serum level at T1;
drugs_nsaid14 = NSAID use (yes/no);
age0 (years); cic_bmi_t4 = BMI;
log10mic-4_q = quartile of MIC-1 serum MIC-1 level at T4;
sm_smokcatg = smoking category (never, ceased, current);
sex (male/female) deltamic1 = T4 serum MIC-1 level-T1 serum MIC-1 level
Note:
(T1 = 1 year after day 0 (T0) when subject is free from polyps, T4 = 4 years after T0). [symbol]
indicates data missing or illegible when filed

### Example Algorithm:

The beta coefficients from the logistic regression for the prediction of polyps in Table A are used to construct the algorithm. The score (Y) is calculated thus:
Y=SEX (for Male=1.3 and for female=1)+{AGE X (−0.02)·}+SMOKING STATUS (for current=1, for past=−0.5, for never=−0.3)+NSAID use (for No=1 and for yes=−0.8)+{MIC-1 LEVEL AT T1 (MIC-1 [pg/ml] X (−0.002)}+{BMI [Kg/m²] X (−0.02))+{CHANGE 1N MIC-1 SERUM LEVEL (T4-T1 [pg/ml] X (0.001)}+{QUARTILE OF log₁₀MIC-1 AT T4 (1^{st}=1, 2^{nd}=1.1, 3^{rd}=2.5 and 4^{th}=3.6)}

So, for the 45 year old male subject mentioned above, the score Y is:
Y=1.3+45(−0.02)+(−0.3)+(−0.8)+{MIC-1 LEVEL AT T1 (MIC-1 [pg/ml] X (−0.002)}+{BMI [Kg/m²] X (−0.02)}+{CHANGE IN MIC-1 SERUM LEVEL (T4-T1 [pg/ml] X (0.001)}+{QUARTILE OF log₁₀MIC-1 AT T4 (1^{st}=1, 2^{nd}=1.1, 3^{rd}=2.5 and 4^{th}=3.6)}

The higher the algorithm score, the more likely it is that a polyp is present in the subject. This is shown in Table B below which provides the relative risk for the presence of one or more polyps, stratified by the MIC-1 score from the algorithm above. This calculation is based on the data for the 317 patients mentioned in Example 1 hereinafter. The range of relative risk is from 1 for subjects in the bottom 0-40% of scores to more than 15 in subjects with scores in the top 20%.

TABLE B

Relative risk of adenoma presence with algorithm score

| Percentile | Score range | OR | LL UL | P-Value |
|---|---|---|---|---|
| 0-20% | −11.19-−1.77 | 1 | Reference | |
| 21-40% | −1.76-−1.31 | 4.52 | 1.41-14.49 | 0.011 |
| 41-60% | −1.30-−0.94 | 3.47 | 1.05-11.43 | 0.041 |
| 61-80% | −0.93-−0.41 | 8.63 | 2.79-26-76 | 0.000 |
| 81-100% | −0.40-0.76 | 13.83 | 4.47-42.74 | 0.000 |

OR = overall risk;
LL = lower limit of risk;
UL = upper limit of risk.

The presence of colorectal polyps may be confirmed, if desired, by methods such as routine colonoscopy, digital rectal examination (DRE), capsule endoscopy, sigmoidoscopy, barium enema or CT colonoscopy.

In the case of a subject who is not a user of NSAIDs, and does not actually have polyps present or, otherwise, has had polyps removed and is considered to be polyp-free following assessment by, for example, routine colonoscopy at a first time point, an elevated change in the amount of MIC-1 at a later (eg second) time point, when adjusted for NSAID use (which in this case, is no NSAID use) and the MIC-1 level at the first time point (eg an initial MIC-1 level), is indicative of an increased likelihood of the development of a polyp or polyp recurrence in the subject. In this subject, the significance of the adjustment based upon the MIC-1 level at the first time point is demonstrated by the finding that:
where the MIC-1 level at the first time point is high (ie above the normal range mentioned above) and the subject is not taking NSAIDs, an elevated change in the amount of MIC-1 at a later time point is actually associated with a reduced likelihood of the development of a polyp or polyp recurrence, whereas
where the MIC-1 level at the first time point is high, no or only a minimal change in the amount of MIC-1 at a later time point is associated with an increased likelihood of the development of a polyp or polyp recurrence, and
where the MIC-1 level at the first time point is within the normal range, an elevated change in the amount of MIC-1 at a later time point is associated with an increased likelihood of the development of a polyp or polyp recurrence.

In the case of a subject who is a user of NSAIDs, and does not actually have polyps present or, otherwise, has had polyps removed and is considered to be polyp-free following assessment by, for example, routine colonoscopy at a first time point, and shows no or only a minimal change in the amount of MIC-1 at a later (eg second) time point, when adjusted for NSAID use and the MIC-1 level at the first time point (eg an initial MIC-1 level), that lack of a change in the amount of MIC-1 is indicative of an increased risk of the development of a polyp or polyp recurrence in the subject. In this subject, the significance of the adjustment based upon NSAID use and the MIC-1 level at the first time point is demonstrated by the finding that:
where the MIC-1 level at the first time point is high and the subject is taking NSAIDs, an elevated change in the amount of MIC-1 at a later time point is associated with an increased likelihood of the development of a polyp or polyp recurrence.

In accordance with the method of the first aspect, the amount of MIC-1 in a test body sample may be determined at different time points (eg at least first and second time points) in the subject: The interval between time points may be determined on a case-by-case basis according to the needs of the subject and may be, for example, three months, six months, one year, three years, five years, ten years or combinations thereof, but it is to be understood that the time intervals may be adjusted according to any relevant health and medical factors of the subject. Preferably, the first time point coincides with the subject being free of colorectal polyps (eg naturally or following removal (eg by polypectomy)). In one preferred embodiment, the time points are as follows:

| | |
|---|---|
| First time point | Day 0/Year 0 (T0) when |
| | subject is free from polyps |
| Second time point | 1 year later (T1) |
| Third time point | 3 years later (T4). |

The change in the amount of MIC-1 in the test body sample may be detected by comparison with a normal subject(s), for example, by
(i) determining the change in the amount of MIC-1 present in the said test body sample; and
(ii) comparing said change against the change or a range of change in the amount of MIC-1 determined from comparative body sample(s) taken from normal subject(s) at the same or substantially equivalent time points;
   such that where, for example, the degree of change in the amount of MIC-1 determined in (i) is greater than that or the range of change determined from the comparative body sample(s) from normal subject(s), there is an elevated amount of change in the amount of MIC-1 that may be associated with the presence of one or more colorectal polyp(s).

In relation to the method of the first aspect, the term “normal subject” refers to a subject who has no colorectal polyps as determined by, for example, colonoscopy and/or barium enema or CT colonoscopy. Preferably, the normal subject(s) is/are also of good health, does/do not smoke and is/are of the same gender as the subject providing the test body sample(s). Further, the normal subject(s) is/are preferably age-matched, wherein the normal subject(s) is/are within 10 years of the age of the subject providing the test body sample(s) and, more preferably, is/are within 5 years of the age of the subject providing the test body sample(s).

As described above, the method of the first aspect enables the change in the amount of MIC-1 to be used as a diagnostic marker of the presence of one or more colorectal polyps or as a prognostic marker of the likelihood of recurrence of colorectal polyps. However, the sensitivity and specificity of such a method may depend on more than just the analytical “quality” of the method; it may also depend on the definition of what constitutes an abnormal result. That is, typically, for any particular marker, the distribution of marker levels for subjects with and without a disease overlaps such that a diagnostic/prognostic test based on that marker will not absolutely distinguish a normal subject from a diseased subject with complete accuracy. Thus, in some embodiments, the method may further comprise calculating receiver operating characteristic (ROC) curves by, for example, plotting the value of the MIC-1 change in amount versus the relative frequency of that value in “normal” and “disease” subject(s). The area under an ROC curve calculated in this manner can then be used as a measure of the probability that the determined change in amount of MIC-1 in the test body sample(s) will allow a correct diagnosis or prognosis. In addition, a ROC curve can even be used where, for whatever reason, the determined change in amount of MIC-1 may be considered as being inaccurate, since so long as it is possible to rank results, a ROC curve can still be calculated; for example, the determined MIC-1 amounts from test body sample(s) from subjects may be ranked according to degree (say 1=low, 2=normal, and 3=high). This ranking can then be correlated to results from normal subject(s) and an ROC curve created according to methods well known to persons skilled in the art (eg Hanley and McNeil, 1982).

The method of the first aspect is preferably conducted in vitro.

In an in vitro method, the amount of MIC-1 present in a test body sample(s) may be readily determined by any suitable method including, for example, immunoassays such as enzyme-linked immunosorbant assay (ELISA), radioimmunoassay (RIA) and immunohistochemistry (eg with sectionalised samples of a tissue biopsy) using anti-MIC-1 antibodies or fragments thereof. However, it is also possible to detect and quantify the levels of MIC-1 using other methods well known to persons skilled in the art such as, for example, methods involving the detection of binding of MIC-1 to a MIC-1 receptor (eg as disclosed in WO 2009/21293) or any other ligands that may bind MIC-1 (eg fetuin as disclosed in WO 2005/99746). Particularly suitable methods for determining the amount of MIC-1 present in a test body sample(s) are immunoassays utilising labelled molecules in various sandwich, competition, or other assay formats. Such immunoassays will develop a signal which is indicative for the presence or absence of MIC-1. Further, the strength of the signal generated by such immunoassays may be correlated directly or indirectly (for example, reversely proportional) to the amount of MIC-1 present in a sample(s). Other particularly suitable methods for determining the amount of MIC-1 present in a test body sample(s) are methods comprising the measurement of a physical or chemical property specific for MIC-1 such as a precise molecular mass or nuclear magnetic resonance (NMR) spectrum. Such methods may, therefore, be conducted using biosensors, optical devices coupled to immunoassays, biochips, analytical devices such as mass-spectrometers, NMR-analysers and chromatography devices. Further particularly suitable methods for determining the amount of MIC-1 present in a test body sample(s) include microplate ELISA-based methods, fully-automated or robotic immunoassays (available, for example, on Elecsys® analysers; Roche Diagnostics Corporation, Indianapolis, Ind., United States of America), enzymatic Cobalt Binding Assay (CBA) (available, for example, on Roche-Hitachi analysers; Roche Diagnostics Corporation) and latex agglutination assays (available, for example, on Roche-Hitachi analysers). Still further examples of particularly suitable methods for determining the amount of MIC-1 present in a test body sample(s) include methods involving precipitation (eg immunoprecipitation), electrochemiluminescence (ie electro-generated chemiluminescence), electrochemiluminescence sandwich immunoassays (ECLIA), dissociation-enhanced lanthanide fluoro immuno assay (DELFIA), scintillation proximity assay (SPA), turbidimetry, nephelometry, latex-enhanced turbidimetry and nephelometry. Further methods that are well known to persons skilled in the art, such as gel electrophoresis, Western Blotting and mass spectrometry, may also be used alone or in combination with other suitable methods as described above.

As such, the determination of the amount of MIC-1 in the test body sample(s) may comprise the steps of (i) contacting MIC-1 with a specific ligand, (ii) optionally removing non-bound ligand, and (iii) measuring the amount of bound ligand. The bound ligand (which may be bound by covalent and/or non-covalent binding) will generate an intensity signal. As indicated above, the ligand may be selected from anti-MIC-1 antibodies or fragments, thereof but might otherwise be selected from any other ligands that may bind MIC-1 such as, for example, any compound (including peptides, polypeptides, nucleic acids, aptamers (eg nucleic acid or peptide aptamers), glycoproteins such as fetuin, and small molecules) that bind to MIC-1. However, preferably, the ligand is selected from anti-MIC-1 antibodies or fragments thereof (including polyclonal and monoclonal antibodies, as well as fragments thereof, such as Fv, Fab and F(ab)₂ fragments that are capable of binding MIC-1, and recombinant antibodies such as single chain antibodies (eg scFV antibodies)) and a MIC-1 receptor (eg as disclosed in WO 2009/21293) or fragment thereof comprising at least one binding domain that binds to MIC-1. Methods of preparing such ligands are well known to persons skilled in the art.

Preferably, the ligand binds specifically to MIC-1. As used herein, the term “specific binding” means that the ligand should not bind substantially to (that is, substantially “cross-react” with) another peptide, polypeptide or substance present in the test body sample. Preferably, the specifically bound MIC-1 will be bound with at least 3 times higher, more preferably at least 10 times higher, and most preferably at least 50 times higher affinity than any other relevant peptide, polypeptide or substance. Non-specific binding may be tolerable, if it can still be distinguished and measured unequivocally, for example, according to its size on a Western Blot, or by the relatively higher abundance of MIC-1 in the sample, or if it can be controlled for using a negative control sample or a normal subject(s) control sample.

The ligand may be coupled covalently or non-covalently to a label allowing detection and measurement of the ligand. Suitable labelling may be performed by any of the direct or indirect methods well known to persons skilled in the art. However, by way of brief explanation, direct labelling involves the coupling of the label directly (ie covalently or non-covalently) to the ligand, while indirect labelling involves the binding (ie covalently or non-covalently) of a secondary ligand to the ligand (ie “primary ligand”) wherein the secondary ligand should specifically bind to the first ligand and may be coupled with a suitable label and/or be the target (receptor) of tertiary ligand binding to the secondary ligand. The use of secondary, tertiary or even higher order ligands can be used to increase the signal. Suitable secondary and higher order ligands may include antibodies, secondary antibodies, and the well-known streptavidin-biotin system (Vector Laboratories, Inc, Burlingame, Calif., United States of America). The ligand may also be “tagged” with one or more tags well known to persons skilled in the art, which tags may then be targets for higher order ligands. Suitable tags include biotin, digoxygenin, His-Tag, glutathione-5-transferase, FLAG, Green Fluorescent Protein (GFP), myc-tag, Influenza A virus haemagglutinin (HA), maltose binding protein and the like. Where the ligand is a protein, peptide or polypeptide, the tag is preferably located at the N-terminus and/or C-terminus. Suitable labels include any labels that are detectable by an appropriate detection method such as, for example, gold particles, latex beads, acridan ester, luminol, ruthenium, enzymatically-active labels, radioactive labels, magnetic labels (for example, “magnetic beads”, including paramagnetic and superparamagnetic labels), and fluorescent labels. Suitable enzymatically-active labels include, for example, horseradish peroxidase, alkaline phosphatase, β-galactosidase, luciferase and derivatives thereof. Suitable substrates for enzymatically-active labels to enable detection include di-amino-benzidine (DAB), 3,3′-5,5′-tetramethylbenzidine, 4-nitro blue tetrazolium chloride and 5-bromo-4-chloro-3-indolyl-phosphate (NBT-BCIP), available as a ready-made stock solution from Roche Diagnostics Corporation), CDP-Star™ (Amersham Biosciences Inc, Fairfield, Conn., United States of America), and ECF™ (Amersham Biosciences Inc). Suitable radioactive labels include ³⁵S, ¹²⁵I, ³²P, ³³P and the like. Radioactive labels can be detected by any of the methods well known to persons skilled in the art including, for example, a light-sensitive film or a phosphor imager. Suitable fluorescent labels include fluorescent proteins (such as GFP and derivatives thereof, Cy₃, Cy₅, Texas Red, Fluorescein and the Alexa dyes (eg Alexa 568)). The use of quantum dots as fluorescent labels is also contemplated.

In some embodiments, the amount of MIC-1 in a test body sample(s) may be determined as follows: (i) contacting a solid support comprising a ligand for MIC-1 as described above with said test body sample(s) comprising MIC-1 and thereafter (ii) measuring the amount of MIC-1 which has become bound to the support. Preferably, in such embodiments, the ligand is selected from the group of ligands consisting of nucleic acids, peptides, polypeptides, antibodies and aptamers, and, preferably, is provided on the solid support in an immobilised form. The solid support may be composed of any of the typical materials well known to persons skilled in the art including, inter alia, commercially available column materials, polystyrene beads, latex beads, magnetic beads, colloid metal particles, glass and/or silicon chips and surfaces, nitrocellulose strips, membranes, sheets, duracytes, wells and walls of suitable reaction trays such as 96-well plates and other plates, plastic tubes etc. The ligand used in such embodiments may also be bound to a suitable carrier such as glass, polystyrene, polyvinyl chloride (PVC), polypropylene, polyethylene, polycarbonate, dextran, nylon, amyloses, natural and modified celluloses, polyacrylamides, agaroses and magnetite. The nature of the carrier can be either soluble or insoluble. Suitable methods for immobilising the ligand to the solid support are well known to persons skilled in the art and include, for example, ionic, hydrophobic, covalent interactions and the like. It is also contemplated to use “suspension arrays” (Nolan and Sklar, 2002), wherein a carrier such as a microbead or microsphere is present in suspension and the array consists of different microbeads or microspheres, possibly labelled, carrying different ligands. Methods of producing such arrays, for example based on solid-phase chemistry and photo-labile protective groups, are well known to persons skilled in the art (see, for example, U.S. Pat. No. 5,744,305).

Where a change in the amount of MIC-1, or a shift in rate of change in the amount of MIC-1, is detected that is associated with the presence of one or more colorectal polyp(s) in the subject, the method may further comprise:

(ii) treating said subject so as to remove, reduce or manage the colorectal polyp(s) that are present.

The step of treating the subject may involve any one or more of the treatments well known to persons skilled in the art including, for example, polypectomy using a snare or biopsy forceps, or partial or total collectomy.

While not wishing to be bound by theory, the present applicants consider that MIC-1 has a role in protecting against the development of colorectal polyps. Accordingly, it is anticipated that MIC-1-enhancing agents may provide the basis of a therapeutic or preventative treatment.

Thus, it is to be understood that the present invention extends to a method for treating or preventing colorectal polyp(s) in a subject, said method comprising administering to said subject an effective amount of an agent selected from the group consisting of macrophage inhibitory cytokine-1 (MIC-1), MIC-1 agonists and MIC-1-enhancing agents, optionally in admixture with a pharmacologically-acceptable carrier and/or excipient.

A “MIC-1 agonist”, for the purposes of the present invention, includes agents which mimic the activity of MIC-1 (eg peptide mimetics of the active domains of MIC-1, and small organic molecules which mimic MIC-1 activity by, for example, binding to and stimulating the activity of the MIC-1 receptor complex). A “MIC-1-enhancing agent”, for the purposes of the present invention, includes any agent that may increase the amount of endogenous MIC-1 in a subject (particularly, the serum level of endogenous MIC-1), and may be selected from agents which enhance transcription or translation of the MIC-1 gene (eg the p53 transcription factor, which is often seen in elevated levels in diseases associated with MIC-1 over-expression, or agents which enhance p53 expression or activity such as nutlin).

An agent for use in the method of treating or preventing colorectal polyp(s), may be formulated into any suitable pharmaceutical/veterinary composition or dosage form (eg compositions for oral, buccal, nasal, intramuscular and intravenous administration). Typically, such a composition will be administered to the subject in an amount which is effective to treat or prevent polyp(s), and may therefore provide between about 0.01 and about 100 μg/kg body weight per day of the agent, and more preferably provide from 0.05 and 25 μg/kg body weight per day of the agent. A suitable composition may be intended for single daily administration, multiple daily administration, or controlled or sustained release, as needed to achieve the most effective results.

As mentioned above, while investigating the association between MIC-1 expression and colorectal polyps, the present applicants recognised that for the use of MIC-1 levels as the basis for diagnostic and/or prognostic methods, it may be at least desirable to account for the effect of certain factors such as age, gender etc on detected MIC-1 levels.

Accordingly, in a second aspect, the present invention provides a method of diagnosing or prognosing a disease or condition in a subject that is associated with increased or over expression of macrophage inhibitory cytokine-1 (MIC-1), the method comprising detecting:

(i) an elevated amount of MICA in a test body sample from said subject, wherein the elevated amount of MIC-1 is associated with said disease or condition;
(ii) a change in the amount of MIC-1 in a test body sample from said subject taken at two or more time points, wherein the elevated change in the amount of MIC-1 is associated with said disease or condition; and/or
(iii) a shift in rate of change in the amount of MIC-1 in a test body sample from said subject taken at two or more time points, wherein the shift in rate of change in the amount of MIC-1 is associated with said disease or condition;
wherein said elevated amount, change in the amount of MIC-1 or shift in rate of change in the amount of MIC-1, is adjusted for the effect of at least the following factors as appropriate: the gender of the subject, the age of the subject, the body mass index (BMI) of the subject, the subject being a smoker, the subject being a user of non-steroidal anti-inflammatory drugs (NSAIDs), and the waist-to-hip ratio where the subject is female.

Preferably, said elevated amount, change in the amount of MIC-1 or shift in rate of change in the amount of MIC-1, is also adjusted for one or more of the following further factors as appropriate: toxic environmental factors, markers of mortality such as interleukin-6 (IL-6), C-reactive protein (CRP) and/or short telomere length (De Meyer et al., 2008), renal function, and/or fat component of body mass index (FBMI), fibrinogen level and/or 8-hydroxydeoxyguanosine (8-OHdG) level, measures of oxidative stress, and dietary factors.

In one form of the invention according to the second aspect, the method comprises detecting:

(i) an elevated amount of MIC-1 in a test body sample from said subject, wherein the elevated amount of MIC-1 is associated with said disease or condition.

The amount of MIC-1 that may be regarded as an “elevated amount” of MIC-1 in this context may vary according to any of a number of factors, for example, the particular body sample type used, the gender of the subject, the age of the subject, the body mass index (BMI) of the subject, the smoking status of the subject, any use of NSAIDs, the waist-to-hip ratio in female subjects, the severity of the disease and/or condition in the subject, when the test body sample is taken, and the method used to detect the elevated amount of MIC-1 and, possibly, the racial origin of the subject, the time of day that the test body sample(s) was taken, and the nature of any exercise undertaken and/or meals consumed by the subject prior to the taking of the test body sample(s). However, in accordance with the method of the second aspect, the elevated amount is adjusted for the effect of at least the following factors as appropriate: the gender of the subject, the age of the subject, the body mass index (BMI) of the subject, the subject being a smoker, the subject being a user of non-steroidal anti-inflammatory drugs (NSAIDs), and the waist-to-hip ratio where the subject is female.

Such adjustment may be made using an algorithm utilising beta coefficients from the logistic regression for the prediction of said disease or condition. As such, persons skilled in the art would understand that the greater the algorithm score, the more likely it is that the subject has the relevant disease or condition.

Alternatively, the elevated amount of MIC-1 for the purposes of the method of the second aspect may be represented by a detected (and adjusted) concentration amount that is greater than a specific pre-determined amount such as, for example, a serum MIC-1 level greater than about 1150 pg/ml (or, in the case of a whole blood or blood plasma sample, a level that corresponds to a serum MIC-1 level greater than about 620 pg/ml; for example, since serum comprises about 50-55% of whole blood, a whole blood MIC-1 level of about 575 pg/ml approximately corresponds to a serum MIC-1 level of about 1150 pg/ml) or, preferably, a serum MIC-1 level greater than about 1200 pg/ml (or, in the case of a whole blood or blood plasma sample, a level that corresponds to a serum MIC-1 level greater than about 1200 pg/ml) or, more preferably, a serum MIC-1 level greater than about 2000 pg/ml (or, in the case of a whole blood or blood plasma sample, a level that corresponds to a serum MIC-1 level greater than about 2000 pg/ml), still more preferably, a serum MIC-1 level greater than 5000 pg/ml (or, in the case of a whole blood or blood plasma sample, a level that corresponds to a serum MIC-1 level greater then about 5000 pg/ml), and most preferably, a serum MIC-1 level greater than about 10000 pg/ml (or, in the case of a whole blood or blood plasma sample, a level that corresponds to a serum MIC-1 level greater than about 10000 pg/ml). The specific pre-determined amount may be identified by comparison with a reference amount of MIC-1 that is known to be associated with said disease or condition: Preferably, the reference amount is an increased amount compared to the amount or a range of amounts of MIC-1 present in comparative body sample(s) taken from normal subject(s).

In another form of the invention according to the second aspect, the method comprises detecting:

(ii) a change in the amount of MIC-1 in a test body sample from said subject taken at two or more time points, wherein the elevated change in the amount of MIC-1 is associated with said disease or condition.

The change in the amount of MIC-1 may also vary according to any of a number of factors, for example, the particular body sample type used, the sex of the subject, the age of the subject, the body mass index (BMI) of the subject, the smoking status of the subject, any use of NSAIDs (particularly in male subjects), the waist-to-hip ratio in female subjects, the severity of the disease and/or condition in the subject, when the test body samples are taken, and the method used to detect the elevated change in amount of MIC-1 and, possibly, the racial origin of the subject, the time of day that the test body samples were taken, and the nature of any exercise undertaken and/or meals consumed by the subject prior to the taking of the test body samples. However, in accordance with the method of the second aspect, the change in the amount of MIC-1 is adjusted for the effect of at least the following factors as appropriate: the gender of the subject, the age of the subject, the body mass index (BMI) of the subject, the subject being a smoker, the subject being a user of non-steroidal anti-inflammatory drugs (NSAIDs), and the waist-to-hip ratio where the subject is female.

Such adjustment may be made using an algorithm based upon multivariate regression (ie in the manner described above).

Thus, for example, in the case of a smoking female test subject of 40 years of age having a known BMI and no history of NSAID use, the elevated amount referred to in (i) is adjusted for the effect of her gender, age and waist-to-hip ratio on MIC-1 expression. Such adjustment may be made using an algorithm utilising beta coefficients from the logistic regression for the prediction of polyp recurrence (see Table C), but persons skilled in the art will understand that the calculation can be readily modified by generating suitable beta coefficients for these factors for the prediction of another disease or condition.

TABLE C

| Logistic regression | | | | Number of obs = 313 | | |
|---|---|---|---|---|---|---|
| | | | | L [symbol] chi2(12) = 51.61 | | |
| | | | | Prob > chi2 = 0.0000 | | |
| Log likelihood = −157. [symbol] 4229 | | | | Pseudo R2 = 0.1410 | | |
| recur4 | Coef. | Std. Err. | x | p > \|x\| | [95% Conf. Interval] | |
| mic_1_pg_m-1 | −.001 [symbol] 601 | .0006613 | −2. [symbol] 1 | 0.005 | −.0031563 | −.000564 |
| 1.drugs_d-14 | −.785 [symbol] 746 | .309 [symbol] 729 | −2.54 | 0.011 | −1.392626 | −.1791229 |
| age0 | −.0195729 | .019402 | −1.01 | 0.313 | −.0576002 | .01845 [symbol] |
| cic_bmi_t4 | −.00 [symbol] 7669 | .0396 [symbol] | −0.17 | 0. [symbol] 65 | −.0 [symbol] 54 [symbol] 1 | .07101 [symbol] 3 |
| log10mic-4_q | | | | | | |
| 1 | 1.105 [symbol] 76 | .5004309 | 2.21 | 0.027 | .1250495 | 2.0 [symbol] 6702 |
| 2 | 2.56016 | .59750 [symbol] | 4.28 | 0.000 | 1.3 [symbol] 90 [symbol] | 3.7312 [symbol] |
| 3 | 3.644891 | . [symbol] 62337 [symbol] | 4.23 | 0.000 | 1. [symbol] 4741 | 5.335041 |
| deltamic1 | −.0011982 | .000 [symbol] 46 | −1. [symbol] 5 | 0.06 [symbol] | −.00246 [symbol] 3 | .0000679 |
| sm_smokcatg | | | | | | |
| 1 | −.479810 [symbol] | .314 [symbol] 404 | −1.52 | 0.12 [symbol] | −1.096 [symbol] 6 | .1372655 |
| 2 | −. [symbol] 29 [symbol] 60 [symbol] | .44 [symbol] 0058 | −0.7 [symbol] | 0.462 | −1.207936 | .5482147 |
| waisttohip | −.850670 [symbol] | 2.11 [symbol] 171 | −0.40 | 0. [symbol] | −5.00221 | 3.300869 |
| 1.sex | 1.31 [symbol] 054 | .4243174 | [symbol] .11 | 0.002 | .4 [symbol] 4073 | 2.1 [symbol] 9701 |
| _ [symbol] | .8514785 | 1.906119 | 0.45 | 0.655 | −2. [symbol] 4447 | 4.5 [symbol] 7 [symbol] |

recur4 = recurrence of polyp at T4;
Coef. = beta coefficients;
mic_1_pg_m-1 = MIC-1 serum level at T1;
1.drugs_d-14 = NSAID use (yes or no);
age0 = age (years);
cic_bmi_t4 = BMI;
log10mic-4_q = quartile of serum MIC-1 level at T4;
deltamic1 = T4-T1 serum MIC-1 level;
sm_smokcatg = smoking category (never, ceased, current);
waisttohip = waist to hip ratio;
1.sex = 1 = male;
Note:
(T1 = 1 year after day 0 (T0) when subject is free from polyps, T4 = 4 years after T0). [symbol]
indicates data missing or illegible when filed

A weighted score (Y) can be calculated thus:
Y=SEX (Male=1.3 and for female=1)+{AGE X (−0.02)}+SMOKING STATUS (for current=1, for past=−0.5, for never=−03)+NSAID use (for No=1 and for yes=−0.8)+MIC-1 LEVEL AT T1 (MIC-1 [pg/ml] X (−0.002))+{BMI [Kg/m²] X (−0.007)}+{CHANGE IN MIC-1 SERUM LEVEL (T4-T1 [pg/ml] X (0.001)}+{QUARTILE OF log₁₀MIC-1 AT T4 (1^{st}=1, 2^{nd}=1.1, 3^{rd}=2.6 and 4^{th}=3.6)}+WAIST TO HIP RATIO X −0.85

So, for the 40 year old female subject mentioned above, the score Y is:
Y=1+40(−0.02)+(1)+(1)+{MIC-1 LEVEL AT T1 (MIC-1 [pg/ml] X (−0.002)}+{BMI [Kg/m²] X (−0.007))+{CHANGE IN MIC-1 SERUM LEVEL (T4-T1 [pg/ml] X (0.001)}+{QUARTILE OF log₁₀MIC-1 AT T4 (1^{st}=1, 2^{nd}=1.1, 3^{rd}=2.5 and 4^{th}=3.6)}+WAIST TO HIP RATIO X −0.85

The higher the algorithm score, the more likely it is that one or more polyp is present in the subject. This is shown in Table D below which shows the relative risk for the presence of one or more polyps, stratified by the MIC-1 score from the algorithm above. The range of relative risk is from about 1 for subjects in the bottom 0-40% of scores to more than 15 in subjects with scores in the top 20%.

TABLE D

Relative risk of adenoma presence with algorithm score

| Percentile | Score range | OR | LL UL | P-Value |
|---|---|---|---|---|
| 0-20% | −11.21-−2.09 | 1 | Reference | |
| 21-40% | −2.08-−1.46 | 1.58 | 0.42-5.90 | 0.496 |
| 41-60% | −1.45-−0.87 | 6.86 | 2.19-21.52 | 0.001 |
| 61-80% | −0.86-−0.28 | 8.70 | 2.79-27.10 | 0.000 |
| 81-100% | −0.27-1.44 | 15.23 | 4.93-47.00 | 0.000 |

OR = overall risk;
LL = lower limit of risk;
UL = upper limit of risk.

Persons skilled in the art would understand that the greater the change in the amount of MIC-1 detected in a subject (ie as represented by an algorithm score), the more likely it is that the subject has the relevant disease or condition.

Accordingly, in this form of the method of the second aspect, the amount of MIC-1 in a test body sample may be determined at different time points (eg at least first and second time points) in the subject. The interval between time points may be determined on a case-by-case basis according to the needs of the subject and may be, for example, three months, six months, one year, three years, five years, ten years or combinations thereof, but it is to be understood that the time intervals may be adjusted according to any relevant health and medical factors of the subject. In one preferred embodiment, the time points are as follows:

| | |
|---|---|
| First time point | Day 0/Year 0 (T0) when subject is free of |
| | any disease or condition associated with |
| | increased or over expression of MIC-1 |
| Second time point | 1 year later (T1) |
| Third time point | 3 years later (T4). |

A change in the amount of MIC-1 in the test body sample may be detected by comparison with a normal subject(s), for example, by
(i) determining the change in the amount of MIC-1 present in the said test body sample; and
(ii) comparing said change against the change or a range of change in the amount of MIC-1 determined from comparative body sample(s) taken from normal subject(s) at the same or substantially equivalent time points;
   such that where, for example, the degree of change in the amount of MIC-1 determined in (i) is greater than that or the range of change determined from the comparative body sample(s) from normal subject(s), there is an elevated amount of change in the amount of MIC-1 that may be associated with the disease or condition.

In relation to the method of the second aspect, the term “normal subject” refers to a subject who does not appear to have any disease or condition associated with increased or over expression of MIC-1. Preferably, the normal subject(s) is/are also of good health, does/do not smoke and is/are of the same sex as the subject providing the test body sample(s). Further, the normal subject(s) is/are preferably age-matched, wherein the normal subject(s) is/are within 10 years of the age of the subject providing the test body sample(s) and, more preferably, is/are within 5 years of the age of the subject providing the test body sample(s).

In still another form of the invention according to the second aspect, the method comprises detecting:

(ii) a shift in rate of change in the amount of MIC-1 in a test body sample from said subject taken at two or more time points, wherein the shift in rate of change in the amount of MIC-1 is associated with said disease or condition.

Where an elevated amount, change in the amount of MIC-1 or shift in rate of change in the amount of MIC-1, is detected that is associated with the disease or condition in the subject, the method of the second aspect may further comprise:

(iii) treating said subject for said disease or condition.

The disease or condition may be selected from, for example, cancers including prostate cancer, breast cancer, colorectal cancer, pancreatic cancer and bladder cancer, cardiovascular disease, atherosclerosis and ischaemic injury, chronic inflammatory diseases including rheumatoid arthritis, fibrotic diseases, chronic kidney disease (especially end-stage renal disease), anorexia/cachexia and other dietary factors, miscarriage risk and/or premature birth, foetal abnormalities, oxidative stress, and environmental toxicity.

As described in WO 2009/052557 (the entire content of which is hereby incorporated by reference), the level of MIC-1 in a test body sample may also be utilised as the basis of a prognostic method of overall survival of an apparently healthy individual.

Thus, in a third aspect, the present invention provides a method of prognosis of overall survival of an apparently healthy subject, the method comprising detecting:

(i) an elevated amount of macrophage inhibitory cytokine-1 (MIC-1) in a test body sample from said subject;
(ii) a change in the amount of MIC-1 in a test body sample from said subject taken at two or more time points; and/or
(iii) a shift in rate of change in the amount of MIC-1 in a test body sample from said subject taken at two or more time points;
wherein said elevated amount or change in the amount of MIC-1 is adjusted for the effect of at least the following factors as appropriate: the gender of the subject, the age of the subject, the body mass index (BMI) of the subject, and the subject being a smoker.

Preferably, said elevated amount, change in the amount of MIC-1 or shift in rate of change in the amount of MIC-1, is also adjusted for the effect of at least the following further factors as appropriate: the subject being a user of non-steroidal anti-inflammatory drugs (NSAIDs), the waist-to-hip ratio where the subject is female.

More preferably, said elevated amount, change in the amount of MIC-1 or shift in rate of change in the amount of MIC-1, is also adjusted for one or more of the following further factors as appropriate: toxic environmental factors, and other markers of mortality such as interleukin-6 (IL-6), C-reactive protein (CRP) and/or short telomere length (De Meyer et al., 2008), and/or fat component of body mass index (FBMI), fibrinogen level and/or 8-hydroxydeoxyguanosine (8-OHdG) level, measures of oxidative stress, and dietary factors.

As used herein, the term “overall survival” is to be understood as referring to the survival of an apparently healthy subject; more particularly, the period that the subject does not die from any cause other than accident or misadventure (eg the subject does not die from a medical cause such as a life-threatening disease or condition such as cancer, particularly an epithelial cancer such as prostate cancer, and cardiovascular disease and events), that is all cause mortality. In other words, “overall survival” refers to the period before the subject dies from all cause mortality.

The term “apparently healthy subject” as used herein, is to be understood as referring to a subject with no apparent symptoms or ill effects of life-threatening diseases or conditions (such as those mentioned above). Preferably, the subject is apparently healthy at the time of taking the test body sample(s) from said subject.

In accordance with the third aspect of the present invention, it is to be understood that the elevated amount, change in the amount of MIC-1 or shift in rate of change in the amount of MIC-1, predicts an increased likelihood of death from any cause other than accident or misadventure (ie the elevated amount, change in amount of MIC-1 or shift in rate of change in the amount of MIC-1, provides a prognosis of the likely death of the apparently healthy subject).

The methods of the second and third aspects are preferably conducted in vitro in the manner as described above in relation to the method of the first aspect.

The test body sample for use in the methods of the second and third aspects may be preferably selected from whole blood, blood plasma, serum, buffy coat, urine, cerebrospinal fluid, seminal fluid, synovial fluid, a tissue biopsy and/or an organ biopsy. More preferably, the test body sample is selected from the group consisting of whole blood, blood plasma, serum and urine. Most preferably, the test body sample is serum.

In accordance with the method of the third aspect, the detection of an elevated amount of MIC-1 is indicative of an increased likelihood of death of the subject, with the possible exception of elderly subjects (eg aged 70 years and above wherein an elevated amount of MIC-1 may be protective from death thereby being indicative of a reduced likelihood of death of the subject). However, in elderly subjects, the detection of an elevated change in the amount of MIC-1 would nevertheless be expected to be indicative of an increased likelihood of death in the subject.

The method of the third aspect may be used in combination with an independent analysis of other markers of mortality such as interleukin-6 (IL-6), C-reactive protein (CRP) and/or short telomere length (De Meyer et al., 2008).

The present invention is hereinafter further described by way of the following, non-limiting example(s).

### EXAMPLES

### Example 1

### Serum MIC-1 for Predicting Recurrence of Colorectal Polyps

The purpose of the study described in this example was three-fold: (i) to examine the relationship between serum MIC-1 concentrations and known risk factors for colorectal cancer risk (ie age, gender, body composition, smoking, diet and NSAID use), (ii) to assess whether serum MIC-1 levels can be utilised as a biomarker of polyp recurrence, and (iii) to determine whether serum MIC-1 levels or the change in MIC-1 levels are predictive of polyp recurrence.

### Methods and Materials

### Study Population

Participating subjects in this study were from the control arm of a Polyp Prevention Trial (PPT), a multicenter randomised clinical trial to evaluate the effects of a high-fibre, high fruit and vegetable, low-fat diet on the recurrence of colorectal polyps. The overall design, rationale, dietary interventions, endpoint procedures, and trial results for the PPT were reported previously (Lanza et al., 1996; Lanza et al., 2001). However, briefly, recruitment activities occurred at eight clinical centres in the United States of America, between 1991 and 1994. Men and women, 35 years or older, were recruited if they had at least one histologically confirmed adenoma removed during a qualifying colonoscopy within 6 months prior to randomisation to either the dietary intervention or control group. Eligible subjects had no history of colorectal cancer, surgical resection of adenomas, bowel resection, polyposis syndrome, or inflammatory bowel disease. In addition, subjects had to be <150% of their recommended weight and could not be currently using lipid-lowering medications. A total of 2079 subjects were enrolled in the PTT, with 1037 randomised to the dietary intervention and 1042 assigned to the control group. The study was completed by 1905 subjects (91.6%), 958 in the intervention group and 947 in the control group. Among the 947 subjects in the control group who completed the PPT, 626 (66.1%) had serum available for the analysis of MIC-1. Three of these subjects were diagnosed with cancer during the study and were therefore excluded, yielding a total of 623 subjects for the analysis.

### Case Ascertainment

Subjects received full colonoscopies at baseline (T0), 1 year (T1), and 4 years after randomisation (T4) at the end of the trial intervention. The colonoscopy at T1 detected and removed any lesions missed at the baseline colonoscopy. Pathologically confirmed polyps diagnosed between T1 and T4 were considered recurrent adenomas. Biopsy samples of all polyps removed during the colonoscopy were reviewed independently by two pathologists to determine the histological features and degree of atypia. Information on the size, number and location of all lesions detected by colonoscopy were obtained from the endoscopy reports. For the analysis, the outcome of “any recurrence” was defined as those subjects who had any recurrence detected by any endoscopic procedure following the 1-year colonoscopy (n=240).

### Statistical Analysis

Statistical analyses were performed using Stata® (Statacorp LP, College Station, Tex., United States of America). Data presented as proportions, such as the baseline characteristics of study subjects stratified by polyp recurrence, were compared by the χ² test. Serum MIC-1 concentrations stratified by covariate data or polyp recurrence were evaluated using the appropriate nonparametric statistical tests (Wilcoxon rank-sum or Kruskal-Wallis tests). Odds ratios (ORs) and 95% confidence intervals (CIs) for adenoma recurrence were estimated within quartiles of serum MIC-1 concentrations. Multivariate logistic regression models included covariates that changed the OR for MIC-1 by >10% or if they were significant predictors of polyp recurrence (p<0.05 using a likelihood ratio test), the final model included age and gender. Effect modification by age and gender was assessed by including the individual factor and its cross-product term with the serum MIC-1 variable in separate multivariate models using the likelihood ratio test. No significant interactions were observed between serum MIC-1 levels and either age or gender. To assess the rate of change in serum MIC-1 levels from T1 to T4 as a predictor of polyp recurrence at T4 in the PPT cohort, a AMIC-1 variable was developed and used in logistic regression modeling of polyp recurrence. In order to evaluate the effect of increasing serum concentrations of MIC-1 independent of surgical intervention, the AMIC-1 analysis was limited to study subjects who were: 1) polyp free at T1, and 2) had blood drawn prior to the T4 colonoscopy/polypectomy procedure or had no polyp recurrence. Additionally, this subgroup analysis was restricted to subjects who had the same NSAID use profile at the time of sampling at T1 and T4 (n=317). All statistical analyses were two-sided and differences were considered significant at P<0.05.

### Results and Discussion

### Population Characteristics

Among the baseline subject characteristics assessed for their relationship with polyp recurrence in the PPT study (Table 1), male gender (P<0.0001), a family history of multiple adenoma (P<0.0001), and elevation in the waist-to-hip ratio (P=0.001) were significantly associated with polyp recurrence at year 4, while a positive association was observed between increased alcohol intake and polyp recurrence (P=0.08). The proportion of regular NSAID users with polyp recurrence at year 4 (65/204=31.9%) was significantly lower than the proportion of non-NSAID users (177/424=41.7%, P=0.02) (Table 1).

### MIC-1 Serum Levels (Polyps Present)

Mean serum MIC-1 levels differed significantly by polyp status with the lowest concentrations in polyp free subjects and the highest concentrations in subjects with polyps present at the time of the blood draw (T1: 827.6 vs. 911.2 pg/ml, P=0.05 and T4: 937.5 vs. 1, 022.1 pg/ml, P=0.03) (Table 2). At T1 and T4, serum MIC-1 concentrations also increased with age (P<0.0001), waist-to-hip ratio (P<0.0001), and in current smokers (P<0.0001) and males (P<0.0001) (Table 2). Conversely, body mass index (body weight in kilograms/height in meters²) was not significantly associated with serum MIC-1 levels (data not shown).

### MIC-1 Serum Levels (NSAID Users)

Serum MIC-1 concentrations were higher among regular NSAID users (P=0.06 for T1 and P<0.0001 for T4) (Table 2). When regular NSAID users were further stratified according to the presence or absence of a polyp at the time of serum MIC-1 estimation, NSAID users at T1 with no polyp present had significantly higher levels of serum MIC-1 (P=0.02). After stratifying by gender (data not shown), the effect of NSAID use on serum MIC-1 levels was more pronounced among men (no regular NSAID use 928.9 vs. regular NSAID use=1, 106.5 pg/ml, P=0.002) than women (no regular NSAID use=834.9 vs. regular NSAID use 894.5 pg/ml, P=0.18). When the remainder of the Table 2 covariates were stratified by gender (data not shown), age and smoking status remained significantly associated with serum MIC-1 concentrations in both men (P<0.0001 and P=0.0003, respectively) and women (P<0.0001 and P=0.03, respectively). Additionally, serum MIC-1 concentrations were increased with elevated waist-to-hip ratio in women (P=0.0005) but not men (P=0.65).

TABLE 1
Characteristics of Participants in Poly Prevention Trial (PPT) by
Adenoma Recurrence^{†}.

| | Total | | No Recurrence | | Recurrence | | |
|---|---|---|---|---|---|---|---|
| Baseline Characteristics | N | % | N | % | N | % | P-value |
| Age | | | | | | | |
| Quartile 1 (35-54) | 152 | 24.20 | 102 | 26.42 | 50 | 20.66 | |
| Quartile 2 (54-62) | 159 | 25.32 | 93 | 24.09 | 66 | 27.27 | |
| Quartile 3 (62-69) | 163 | 25.96 | 104 | 26.94 | 59 | 24.38 | |
| Quartile 4 (69-86) | 154 | 24.52 | 87 | 22.54 | 67 | 27.69 | P = 0.1372 |
| Sex | | | | | | | |
| Male | 385 | 61.31 | 211 | 54.66 | 174 | 71.9 | |
| Female | 243 | 38.69 | 175 | 45.34 | 68 | 28.1 | P < 0.0001 |
| Race | | | | | | | |
| Caucasian | 577 | 91.88 | 357 | 92.49 | 220 | 90.91 | |
| Other | 51 | 8.12 | 29 | 7.51 | 22 | 9.09 | P = 0.6559 |
| Waist to Hip Ratio | | | | | | | |
| Tertile 1 (0.61-0.91) | 205 | 33.23 | 147 | 38.89 | 58 | 24.27 | |
| Tertile 2 (0.91-0.98) | 206 | 33.39 | 115 | 30.42 | 91 | 38.08 | |
| Tertile 3 (0.98-1.51) | 206 | 33.39 | 116 | 30.69 | 90 | 37.66 | P = 0.0014 |
| Smoking History | | | | | | | |
| No | 551 | 87.74 | 339 | 87.82 | 212 | 87.6 | |
| Yes | 77 | 12.26 | 47 | 12.18 | 30 | 12.4 | P = 0.9347 |
| Family History of CRC | | | | | | | |
| No | 455 | 72.45 | 283 | 73.32 | 172 | 71.07 | |
| Yes | 173 | 27.55 | 103 | 26.68 | 70 | 28.93 | P = 0.5409 |
| History of Multiple Adenoma | | | | | | | |
| No | 410 | 65.29 | 283 | 73.32 | 127 | 52.48 | |
| Yes | 218 | 34.71 | 103 | 26.68 | 115 | 47.52 | P < 0.0001 |
| Education Status | | | | | | | |
| <=High School | 155 | 24.68 | 97 | 25.13 | 58 | 23.97 | |
| >High School | 473 | 75.32 | 289 | 74.87 | 184 | 76.03 | P = 0.1080 |
| Use of NSAIDs | | | | | | | |
| No | 424 | 67.52 | 247 | 63.99 | 177 | 73.14 | |
| Yes | 204 | 32.48 | 139 | 36.01 | 65 | 26.86 | P = 0.0173 |
| Alcohol Intake (grams per day) | | | | | | | |
| None | 249 | 39.65 | 164 | 42.89 | 85 | 35.12 | |
| 0.30 to 3.99 | 131 | 20.86 | 78 | 20.21 | 53 | 21.9 | |
| 4.00 to 12.99 | 110 | 17.52 | 65 | 16.84 | 45 | 18.6 | |
| 13.00 to 139.00 | 138 | 21.97 | 79 | 20.47 | 59 | 24.38 | P = 0.0812 |

^{†}Any adenoma recurrence at T4 vs. no adenoma recurrence at T4.

### MIC-1 Serum Level Predicts Polyp Presence and Recurrence

To determine if serum MIC-1 level could predict the presence of a polyp at T1, serum MIC-1 levels as above or below the median were stratified into haptiles. The higher serum MIC-1 haptile was associated with polyp presence at T1 (P=0.01, OR 1.8 95% CI 1.1-2.7). This association was attenuated when adjusted for family history of polyps, NSAID during the preceding year, age, sex, waist-to-hip ratio BMI and alcohol use (P=0.06, OR 1.6 95% CI 1.0-2.8). Similarly, the higher serum MIC-1 haptile was associated with polyp presence at T4 (P=0.003, OR 1.6 95% CI 1.2-2.3). Again, this association was attenuated when adjusted for family history of polyps, NSAID during the preceding year, age, sex, waist-to-hip ratio BMI and alcohol use (P=0.07, OR 1.5 95% CI 1.0-2.2).

However, as MIC-1 may be produced by polyps themselves and play a role in protecting against their development, adjustment of the serum MIC-1 for these factors was considered to determine whether it improved the predictive capacity for serum MIC-1 to identify subjects with polyp recurrence. It was reasoned that the protective nature of MIC-1 would be reflected by the serum level of MIC-1 at T1 and the change in serum level over the three years between T1 and T4. If MIC-1 were protective, a higher serum MIC-1 level at T1 might reflect a lower risk of future polyp recurrence. Additionally, it was likely that changes in serum MIC-1 over that time would interact with the ability of the serum MIC-1 level estimation at T4 to predict polyp recurrence. In addition, an adjustment was made for BMI as serum MIC-1 is related to BMI in other cohorts, which is also related to the development of colonic cancer as well as other factors associated with serum MIC-1 levels or polyp recurrence. When T4 serum MIC-1 level was examined in this way (Tables 3-5), subjects in the highest quartile of serum MIC-1 were found to have a three-fold increased risk of polyp recurrence at T4 (OR 3.531 95% CI 1.378-9.048). While this result indicated that serum MIC-1 estimation was capable of predicting the recurrence of polyps in the total cohort, it was considered whether this result could have been significantly attenuated by the timing of MIC-1 estimation in relation to polyp removal and the changing status of NSAID usage throughout the study.

In about half of the subjects examined, serum MIC-1 level was taken while a polyp was present at T1 and/or after a recurrent polyp was removed at T4, or where the subjects NSAID usage had changed. These subjects' results would have significantly attenuated the capacity of serum MIC-1 to identify subjects with recurrent polyps. As serum MIC-1 levels are affected by the presence of a polyp and NSAID usage, examination was restricted to subjects who had no polyp present at T1, comprising subjects who had no polyp present or where they had had serum MIC-1 determination, after polyp removal. These subjects were further restricted to those that either had no recurrence at T4 or had their recurrent polyp present at blood sampling. Additionally, subjects were selected such that they had the same NSAID use at T1 and T4. In these 317 subjects, there was no difference in serum MIC-1 levels in subjects who had no polyp or where a polyp was removed at T1 (975.2 vs. 990.4 pg/ml, P=0.79). To assess whether the correlation between serum MIC-1 levels at T1, T4 and change in MIC-1 level might lead to difficulty in interpreting the multivariate logistic regression presented in Table 5, the analysis was examined further to determine if this approach was valid.

There are several factors that potentially influence serum MIC-1. In animal models, MIC-1 protects against the formation of polyps. Indeed, in such models (eg APC^{min}; Baek et al., 2006) MIC-1 mediates the effect of the NSAID protection against polyp formation (Baek et al., 2006; Zimmers et al., 2009). Additionally, in humans, there is evidence that serum MIC-1 levels rise with the presence of polyps because MIC-1 is produced by polyps (Brown et al., 2003). Further, serum MIC-1 levels rise with age and are significantly different between men and women of the same age (unpublished data). Additionally, serum MIC-1 levels might be influenced by BMI (Johnen et al., 2007). These findings in animals and humans have several implications for the data in this study. After initial polyp removal, high levels of serum MIC-1 might be expected to protect against polyp recurrence. Additionally, if MIC-1 mediates. NSAID protection from polyp recurrence, subjects who take NSAIDs and do not get a rise in serum MIC-1 levels would be expected to have attenuated protection from NSAID use.

In the population selected for concordant NSAID use and appropriate polyp status at T1 and T4 for serum MIC-1 sampling (n=317), serum MIC-1 was significantly higher in subjects taking NSAIDs (904.1 vs. 1106.0 pg/ml; P<0.01; Mann-Whitney-U test). Additionally, when stratifying the effect of NSAID use on the risk of polyp recurrence by serum MIC-1 level (normal or elevated) at T1, there was no protective effect of NSAIDs (<1200 pg/ml; n=245, P=0.59; chi-square analysis) when serum MIC-1 level was not elevated at T1 (less than or equal to 1200 pg/ml; n=245). However, in subjects with elevated serum MIC-1 levels (n=72), NSAIDs had a significant protective effect (n=72; p<0.01). This indicates that polyp-free MIC-1 serum level identifies subjects who will be protected from polyp formation by NSAID use. Further, the findings indicate that MIC-1 may mediate some of the protective effect of NSAIDs.

In subjects not taking NSAIDs (n=201), protection from polyp recurrence by an elevated serum MIC-1 level (greater than 1200 pg/ml) was not significant (p=0.080); chi-square analysis). However, in the subjects who did not have recurrence there was a higher proportion of subjects with serum MIC-1 levels in the 90^{th} percentile with serum MIC-1 greater than 2000 pg/ml, possibly suggesting a protective effect. These findings indicated that when considering polyp recurrence, the initial, polyp free serum MIC-1 level and the use of NSAIDs should be taken into account in addition to age, BMI at the time of measuring MIC-1 and sex. However, as serum MIC-1 levels are strongly associated with age and therefore will be expected to rise over the course of the three years of the study, the serum MIC-1 levels at these two time points will be significantly and highly correlated, independent of the change in serum MIC-1, which is less than 25% of the T1 value in more than two thirds of subjects (n=212). This leads to the question as to whether change in serum MIC-1 levels alter the predictive power of serum MIC-1 estimation. Unfortunately, it is impossible to determine whether it is valid to include the change in serum MIC-1 level as the combination of MIC-1 level at T1 and T4 and change in serum MIC-1 level over these time points is perfectly correlated unless a validation cohort can be examined. To limit the potential confounding effects of having two highly correlated variables, serum MIC-1 level at T1 and T4, in the same logistic regression, the quartiles of the T4 serum MICA level were adjusted for the prediction of the presence of polyp recurrence using normal or elevated serum MIC-1 level (as above), NSAID use, sex and age. This indicated that an adjusted serum MIC-1 level in the highest quartile at T4 carried an adjusted OR of 7.6 (95% CI 2.5-23.2; Table 6).

When follow-up serum MIC-1 levels at T4 were adjusted for the potential protective effects of serum MIC-1 level as a continuous variable, as well as NSAID use there was there was a modest increase in the predictive power of serum MIC-1 level at T4 for the presence of recurrent polyps (p<0.0001: Table 7). Adjusted T4 serum MIC-1 level in the selected group of 317 subjects (Table 6) showed those in the highest quartile of serum MIC-1 had a more than seven-fold increased risk of polyp recurrence (OR 7.58 95% CI 2.47-23.21). Further, using this adjusted serum T4 serum MIC-1 measurements, receiver operator curve (ROC) analysis revealed an area under the curve (AUC) of 0.73 (95% CI 0.66-0.79) for the prediction of recurrent polyps. The exclusion of MIC-1 serum data significantly lowered this AUC (0.67; 95% CI 0.60-0.73: P=0.02).

The predictive power of the adjusted serum MIC level at T4 was dramatically increased with the inclusion of change in serum MIC-1 between T4 and T1 (Table 8). Here, subjects with a serum MIC-1 level in the top quartile at T4 had more than a 30-fold increased risk of a polyp recurrence (OR 37.2; 95% CI 7.1-195.8).

In any case, it appears that serum MIC-1 level is at least partly responsible for the protective effects of NSAIDs in relation to colorectal polyp recurrence in humans. Additionally, the adjustment of serum MIC-1 levels clearly improves the predictive power of serum MIC-1 levels for the detection of colorectal polyps. Further, serial measurement of serum MIC-1 over time, adjusted for factors such as the initial serum MIC-1 level in the absence of colorectal polyps, may also improve the prediction of colorectal polyps with follow-up serum MIC-1 estimation. Finally, the inclusion of the change in serum MIC-1 level may dramatically increase the predictive power of serial serum MIC-1 estimation.

TABLE 2
Geometric Mean of Serum MIC-1 Levels at T1 and T4 by Patient Characteristics.

| | Serum MIC-1 Levels at T1 (pg/ml) | | | | | Serum MIC-1 Levels at T4 (pg/ml) | | | | |
|---|---|---|---|---|---|---|---|---|---|---|
| Patient Characteristics | N | % | Mean | SEM | P-value | N | % | Mean | SEM | P-value |
| Total | 623 | 100 | 847.86 | 15.94 | | 623 | 100 | 948.73 | 19.47 | P < 0.0001 |
| Polyp Status | | | | | | | | | | |
| No Polyp | 424 | 68.06 | 827.61 | 19.72 | | 424 | 68.06 | 937.48 | 23.85 | |
| Polyp Removed | 97 | 15.57 | 875.41 | 36.19 | | 88 | 14.13 | 914.75 | 52.44 | |
| Polyp Present | 102 | 16.37 | 911.19 | 38.52 | P = 0.0532 | 111 | 17.82 | 1,022.13 | 42.56 | P = 0.0263 |
| Age | | | | | | | | | | |
| Quartile 1 (35-54) | 150 | 24.08 | 574.92 | 16.93 | | 150 | 24.08 | 607.67 | 19.56 | |
| Quartile 2 (54-62) | 159 | 25.52 | 771.21 | 24.18 | | 159 | 25.52 | 857.21 | 27.80 | |
| Quartile 3 (62-70) | 163 | 26.16 | 994.74 | 30.70 | | 163 | 26.16 | 1,164.78 | 40.97 | |
| Quartile 4 (70-86) | 151 | 24.24 | 1,159.70 | 38.30 | P < 0.0001 | 154 | 24.24 | 1,316.90 | 45.03 | P < 0.0001 |
| Sex | | | | | | | | | | |
| Male | 382 | 61.32 | 906.17 | 22.58 | | 382 | 61.32 | 1,010.53 | 26.70 | |
| Female | 241 | 38.68 | 763.02 | 20.61 | P < 0.0001 | 241 | 38.68 | 858.43 | 27.11 | P < 0.0001 |
| Waist to Hip Ratio | | | | | | | | | | |
| . | 10 | 1.61 | | | | 10 | 1.61 | | | |
| Tertile 1 (0.61-0.91) | 204 | 32.74 | 739.97 | 22.48 | | 204 | 32.74 | 818.49 | 28.78 | |
| Tertile 2 (0.91-0.98) | 205 | 32.91 | 877.16 | 29.25 | | 205 | 32.91 | 1,008.08 | 35.37 | |
| Tertile 3 (0.98-1.51) | 204 | 32.74 | 934.70 | 30.36 | P < 0.0001 | 204 | 32.74 | 1,023.21 | 36.04 | P < 0.0001 |
| Smoking Status | | | | | | | | | | |
| Never or Never Regular | 257 | 41.25 | 761.19 | 21.91 | | 257 | 41.25 | 859.33 | 26.13 | |
| Former | 289 | 46.39 | 884.50 | 24.24 | | 289 | 46.39 | 984.67 | 30.43 | |
| Current | 77 | 12.36 | 1,036.71 | 49.46 | P < 0.0001 | 77 | 12.36 | 1,148.16 | 62.29 | P < 0.0001 |
| Regular NSAID Use^{a} | | | | | | | | | | |
| . | 3 | 0.48 | | | | 1 | 0.16 | | | |
| No | 386 | 61.96 | 821.11 | 19.39 | | 359 | 57.62 | 885.49 | 24.08 | |
| Yes | 234 | 37.56 | 890.96 | 27.71 | P = 0.0554 | 263 | 42.22 | 1,038.00 | 31.41 | P < 0.0001 |

^{a}Regular NSAID use (>1 per month) vs. no regular NSAID use (<1 per month) reported at years 1 (T1) and 4 (T4), respectively.

TABLE 3
Geometric Mean of Serum MIC-1 levels at T1 and T4 by Adenoma Recurrence at Year 4.

| | Serum MIC-1 Levels at T1 (pg/ml) | | | | | Serum MIC-1 Levels at T4 (pg/ml) | | | | |
|---|---|---|---|---|---|---|---|---|---|---|
| Baseline Characteristics | N | % | Mean | SEM | P-value | N | % | Mean | SEM | P-value |
| Adenoma Recurrence^{a} | | | | | | | | | | |
| No Adenoma Recurrence | 383 | 61.48 | 827.75 | 21.03 | | 383 | 61.48 | 928.15 | 25.32 | |
| Adenoma Recurrence | 240 | 38.52 | 880.96 | 23.83 | P = 0.0311 | 240 | 38.52 | 982.53 | 30.10 | P = 0.0254 |
| Multiple Adenoma Recurrence^{b} | | | | | | | | | | |
| No Adenoma Recurrence | 383 | 78.97 | 827.75 | 21.03 | | 383 | 78.97 | 928.15 | 25.32 | |
| Multiple Adenoma Recurrence | 102 | 21.03 | 956.46 | 37.32 | P = 0.0019 | 102 | 21.03 | 1078.49 | 46.01 | P = 0.0006 |
| Number of Recurrent Adenomas^{c} | | | | | | | | | | |
| 0 | 383 | 61.48 | 827.75 | 21.03 | | 383 | 61.48 | 928.15 | 25.32 | |
| 1 | 138 | 22.15 | 829.01 | 30.20 | | 138 | 22.15 | 917.14 | 38.64 | |
| 2 | 53 | 8.51 | 906.67 | 47.36 | | 53 | 8.51 | 1016.99 | 59.30 | |
| 3 | 29 | 4.65 | 986.24 | 80.43 | | 29 | 4.65 | 1092.52 | 96.27 | |
| 4 | 10 | 1.61 | 1024.15 | 115.76 | | 10 | 1.61 | 1287.01 | 182.08 | |
| 5 | 4 | 0.64 | 843.16 | 116.01 | | 4 | 0.64 | 971.90 | 102.25 | |
| 6 | 2 | 0.32 | 1342.72 | 269.41 | | 2 | 0.32 | 1115.26 | 92.99 | |
| 8 | 2 | 0.32 | 1342.30 | 510.86 | | 2 | 0.32 | 1472.30 | 336.08 | |
| 10 | 2 | 0.32 | 1173.06 | 352.63 | P = 0.0614 | 2 | 0.32 | 1527.50 | 214.88 | P = 0.0352 |
| Advanced Adenoma Recurrence^{d} | | | | | | | | | | |
| No Adenoma Recurrence | 383 | 92.29 | 827.75 | 21.03 | | 383 | 92.29 | 928.15 | 25.32 | |
| Advanced Adenoma Recurrence | 32 | 7.70 | 939.45 | 83.08 | P = 0.0698 | 32 | 7.70 | 1061.16 | 94.25 | P = 0.0719 |
| High Risk Adenoma Recurrence^{e} | | | | | | | | | | |
| No Adenoma Recurrence | 383 | 85.11 | 827.75 | 21.03 | | 383 | 85.11 | 928.15 | 25.32 | |
| High Risk Adenoma Recurrence | 67 | 14.89 | 971.22 | 55.52 | P = 0.0038 | 67 | 14.89 | 1104.76 | 65.55 | P = 0.0022 |

^{a}Participants who had any andenoma recurrence detected by any endoscopic procedure following the 1-year colonoscopy (n = 240).
^{b}Participants with >1 adenoma identified at defined intestinal sites during their follow-up endoscopic procedure (n = 102).
^{c}The number of recurrent andenomas per patient detected by any endoscopic procedure following the 1-year colonoscopy.
^{d}Adenoma recurrence defined by 1 or 3 criteria: 1) diameter ≧1 cm, 2) high-grade dysplasia, or 3) >25% villous elements (n = 32).
^{e}Participants having either advanced recurrence^{d} or >2 recurrnet polyps detected by endoscopy following the 1-year colonoscopy (n = 67).

TABLE 4
Risk of Adenoma Recurrence by Quartiles of Serum MIC-1 Levels (pg/mL).

| | Any Adenoma Recurence^{a} | | | | | | | | | | | |
|---|---|---|---|---|---|---|---|---|---|---|---|---|
| | | | | | Stratified by Regular NSAID Use^{d} | | | | | | | |
| | Total (n = 623) | | | | No NSAID Use (n = 421) | | | | NSAID Use (n = 202) | | | |
| | 95% CI | | | | 95% CI | | | | 95% CI | | | |
| Regression Models | OR | LL | UL | P-Value | OR | LL | UL | P-Value | OR | LL | UL | P-Value |
| Univariate Model^{b} | | | | | | | | | | | | |
| Quartile 2 (612-831 pg/mL) | 1.245 | 0.778 | 1.993 | 0.360 | 1.284 | 0.741 | 2.225 | 0.374 | 1.167 | 0.468 | 2.909 | 0.741 |
| Quartile 3 (832-1158 pg/mL) | 1.761 | 1.108 | 2.797 | 0.017 | 2.000 | 1.143 | 3.498 | 0.015 | 1.513 | 0.645 | 3.550 | 0.341 |
| Quartile 4 (1159-6520 pg/mL) | 1.505 | 0.945 | 2.396 | 0.085 | 1.709 | 0.982 | 2.975 | 0.058 | 1.225 | 0.509 | 2.946 | 0.650 |
| Multivariate Model^{b,c} | | | | | | | | | | | | |
| Quartile 2 (612-831 pg/mL) | 1.058 | 0.633 | 1.768 | 0.830 | 1.096 | 0.600 | 2.000 | 0.766 | 0.838 | 0.304 | 2.310 | 0.733 |
| Quartile 3 (832-1158 pg/mL) | 1.442 | 0.858 | 2.423 | 0.167 | 1.620 | 0.871 | 3.010 | 0.127 | 1.103 | 0.415 | 2.932 | 0.845 |
| Quartile 4 (1159-6520 pg/mL) | 0.974 | 0.543 | 1.748 | 0.929 | 1.108 | 0.561 | 2.189 | 0.768 | 0.626 | 0.195 | 2.012 | 0.432 |

^{a}Any adenoma recurrence at T4 (n = 240) vs. no adenoma recurrence at T4 (n = 383).
^{b}Reference group is serum MIC-1 quartile 1 (195-611 pg/mL).
^{c}Logistic regression analysis adjusted for age and gender.
^{d}Regular NSAID use (>1 per month, n = 202) vs. no regular NSAID use (<1 per month, n = 421) reported at baseline (T0).

TABLE 5
Risk of Adenoma Recurrence by Quartiles of serum MIC-1 levels
at T4 adjusted for the protective effect of MIC-1

| | Any Adenoma Recurrence (n = 623)^{a} | | | |
|---|---|---|---|---|
| Regression Model | OR | LL | UL | P-Value |
| Univariate model (see table 4) | | | | |
| Multivariate Model^{abc} | | | | |
| Quartile 2 (612-831 pg/mL) | 1.259 | 0.720 | 2.201 | 0.419 |
| Quartile 3 (832-1158 pg/mL) | 2.382 | 1.233 | 4.600 | 0.010 |
| Quartile 4 (1159-6520 pg/mL) | 3.531 | 1.378 | 9.048 | 0.009 |

^{a}Any adenoma recurrence at T4 (n = 240) vs. no recurrence at T4 (n = 383)
^{b}Reference group is MIC-1 quartile1 (195-611 pg/mL)
^{c}Logistic regression adjusted for age, gender, serum MIC-1 level at T1 (continuous), BMI at T1 and T4 (continuous) change in MIC-1 (T4-T1; continuous) and regular NSAID usage at T1 and/or T4 (>1 per month n = 202) vs. no regular NSAID usage (<1 per month n = 421) alcohol use at T1 and T4, amount of NSAID and alcohol used at T1 and T4 and smoking category.

TABLE 6
Risk of Adenoma Recurrence by Quartiles of serum MIC-1 levels at
T4 adjusted for the protective effect of MIC-1 in subjects sampled
at appropriate time in relation to polyp presence at T1 and T4

| | Any Adenoma Recurrence (n = 317)^{a} | | | |
|---|---|---|---|---|
| Regression Model | OR | LL | UL | P-Value |
| Univariate model | | | | |
| Quartile 2 (612-831 pg/mL) | 1.235 | 0.557 | 2.738 | 0.603 |
| Quartile 3 (832-1158 pg/mL) | 2.749 | 1.305 | 5.794 | 0.008 |
| Quartile 4 (1159-6520 pg/mL) | 2.393 | 1.150 | 4.981 | 0.020 |
| Multivariate Model | | | | |
| Quartile 2 (612-831 pg/mL) | 1.756 | 0.714 | 4.317 | 0.220 |
| Quartile 3 (832-1158 pg/mL) | 4.738 | 1.882 | 11.929 | 0.001 |
| Quartile 4 (1159-6520 pg/mL) | 7.576 | 2.473 | 23.210 | 0.000 |
| Age (year) | 0.976 | 0.943 | 1.011 | 0.183 |
| Sex (male) | 3.208 | 1.778 | 5.792 | 0.000 |
| MIC-1 level- T1 (≧1200 pg/ml) | 0.399 | 0.170 | 0.936 | 0.035 |
| NSAID use (yes) | 0.442 | 0.244 | 0.800 | 0.007 |

^{a}Any adenoma recurrence at T4 (n = 85) vs. no recurrence at T4 (n = 232)

TABLE 7
Risk of Adenoma Recurrence by Quartiles of serum MIC-1 levels at
T4 adjusted for the protective effect of MIC-1 in subjects sampled
at appropriate time in relation to polyp presence at T1 and T4

| Regression Model | Any Adenoma Recurrence (n = 317)^{a} | | | |
|---|---|---|---|---|
| Multivariate Model^{abc} | OR | LL | UL | P-Value |
| Quartile 2 (612-831 pg/mL) | 2.114 | 0.858 | 5.369 | 0.103 |
| Quartile 3 (832-1158 pg/mL) | 6.627 | 2.470 | 17.781 | 0.000 |
| Quartile 4 (1159-6520 pg/mL) | 12.279 | 3.470 | 43.452 | 0.000 |

^{a}Any adenoma recurrence at T4 (n = 85) vs. no recurrence at T4 (n = 232)
^{b}Reference group is MIC-1/GDF15 quartile1 (195-611 pg/mL)
^{c}Logistic regression adjusted for age, gender, serum MIC-1/GDF15 level at T1 (continuous), and regular NSAID usage at T1 and T4 (>1 per month n = 116) vs. no regular NSAID usage T1 and T4 (<1 per month n = 201)

TABLE 8
Risk of Adenoma Recurrence by Quartiles of serum MIC-1 levels
at T4 adjusted for the protective effect of MIC-1 (initial
and change in serum levels) in subjects sampled at appropriate
time in relation to polyp presence at T1 and T4

| Regression Model | Any Adenoma Recurrence (n = 317)^{a} | | | |
|---|---|---|---|---|
| Multivariate Model^{abc} | OR | LL | UL | P-Value |
| Quartile 2 (612-831 pg/mL) | 2.757 | 1.061 | 7.164 | 0.037 |
| Quartile 3 (832-1158 pg/mL) | 11.037 | 3.578 | 34.047 | 0.000 |
| Quartile 4 (1159-6520 pg/mL) | 37.231 | 7.079 | 195.802 | 0.000 |

^{a}Any adenoma recurrence at T4 (n = 85) vs. no recurrence at T4 (n = 232)
^{b}Reference group is MIC-1/GDF15 quartile1 (195-611 pg/mL)
^{c}Logistic regression adjusted for age, gender, serum MIC-1/GDF15 level at T1 (continuous), change in serum MIC-1 level between T1 and T4 (continuous) and regular NSA1D usage at T1 and T4 (>1 per month n = 116) vs. no regular NSAID usage T1 and T4 (<1 per month n = 201)

### Example 2

### Improved Disease Diagnosis or Prognosis Based on Serum MIC-1 Levels

As outlined above, there are some limitations in the use of serum MIC-1 estimation for disease diagnosis and prognosis due to its presence in all subjects and its variation with factors such as NSAID use, gender and age. However, when factors that are related to serum MIC-1 levels are accounted for, the predictive power of algorithms including serum MIC-1 improves significantly. Additionally, the study in Example 1 has demonstrated that the change in serum MIC-1 levels over time greatly improves the predictive power in disease.

These factors suggest that an initial serum MIC-1 level has an improved diagnostic capacity when adjusted for factors that alter serum MIC-1 levels in the disease population being investigated. Change in serum MIC-1 levels and/or adjustment of a follow-up serum MIC-1 level might have the capacity to significantly improve the diagnostic use of serum MIC-1 estimation. In this case, the follow-up serum MIC-1 level would be adjusted by factors that vary serum MIC-1 concentration in the disease population. This could include, but not be limited to NSAID use, BMI, age, gender, initial serum MIC-1 level, change in serum MIC-1 level, measures of oxidative stress, measures of systemic inflammation and measures of other disease specific markers, the presence of intercurrent disease (eg diabetes), racial grouping, recent exercise and food intake. This approach would be expected to lead to improved sensitivity and specificity for serum MIC-1 estimation to detect a specific disease.

In the case of colorectal polyps, initial serum MIC-1 levels are capable of detecting the presence of colorectal polyps with serum MIC-1 levels in the top quartile having more than two-fold increased risk of having a polyp (OR 2.4 95% CI 1.1-5.0; p=0.02); which resulted in the detection of 28 polyps of the 85 that were present. When this is adjusted for age, NSAID use and gender, the predictive power of serum MIC-1 almost doubles (OR 4.4 95% CI 1.6-11.7; p=0.0004). However, when this serum MIC-1 level is further adjusted for a previous serum MIC-1 level where there is no polyp, the predictive power of serum MIC-1 level more than doubles again (OR 12.3 95% CI 3.5-43.5; p<0.0001). This is likely due to MIC-1 not only being produced by polyps, but also protecting from them. Additionally, the data shown in Example 1 suggests that MIC-1 mediates the protection afforded by NSAIDs against the development of colorectal polyps. This implies that not only the initial polyp free serum MIC-1 level, but the NSAID adjusted change in serum MIC-1 level might further increase the diagnostic capacity of serial serum MIC-1 level determination for colorectal poly recurrence. With these data, additional adjustment for change in serum MIC-1 level leads to an exponential rise in the predictive capacity of serum MIC-1 level (OR 37.2 95% CI 7.1-195; p<0.0001).

When using the final adjusted serum MIC-1 level as a screening test to detect more than 90% of polyps it performs significantly better than MIC-1 alone. For unadjusted serum MIC-1 level determination to predict more than 90% of polyps the cutoff is 620 pg/ml which is about the middle of the normal range. This leads to poor specificity. However, when using algorithms that include MIC-1 serum levels, the specificity doubles. Consequently, while the use of unadjusted serum MIC-1 might be capable of reducing initial and follow-up colonoscopies by more than 20% (22.8%), the use of serial serum MIC-1 levels three years apart and algorithms including the initial polyp-free level, and change in the amount of MIC-1, as above has the capacity to reduce follow-up colonoscopies by more than 45% (45.6%) which is a significant improvement (p<0.0001; McNemar's test). Therefore, this analysis demonstrates the additional use of serial serum MIC-1 levels and its adjustment for factors affecting these levels can significantly improve the diagnostic capacity of serum MIC-1 estimation. Thus, at a sensitivity level where 90% of polyps are detected, the number of required colonoscopies is approximately halved, which is a big reduction for an expensive and invasive procedure.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are therefore to be considered in all respects as illustrative and not restrictive.

### REFERENCES

Baek, S J et al. Cyclooxygenase inhibitors regulate the expression of a TGF-beta superfamily member that has proapoptotic and antitumorigenic activities. Mol Pharmacol 59: 901-908 (2001).
Baek, S J et al. Nonsteroidal anti-inflammatory drug-activated gene-1 over expression in transgenic mice suppresses intestinal neoplasia. Gastroenterology 131:1553-1560 (2006).
Bauskin, A et al. The propeptide of macrophage inhibitory cytokine (MIC-1), a TGF-beta superfamily member, acts as a quality control determinant for correctly folded MIC-1. Embo J 19:2212-2220 (2000).
Bauskin, A R et al. Role of macrophage inhibitory cytokine-1 in tumourigenesis and diagnosis of cancer. Cancer Res 66(10):4983-4986 (2006).
Bootcov, M R et al. MIC-1, a novel macrophage inhibitory cytokine, is a divergent member of the TGF-beta superfamily. Proc Natl Acad Sci USA. 94(21):11514-9 (1997).
Bottner, M et al. Expression of a novel member of the TGF-beta superfamily, growth/differentiation factor-15/macrophage-inhibiting cytokine-1 (GDF-15/MIC-1) in adult rat tissues. Cell Tissue Res 297(1):103-110 (1999a).
Bottner, M et al. Characterization of the rat, mouse, and human genes of growth/differentiation factor-15/macrophage inhibiting cytokine-1 (GDF-15/MIC-1). Gene 237: 105-111 (1999b).
Brown, D A et al. Concentration in plasma of macrophage inhibitory cytokine-1 and risk of cardiovascular events in women: a nested case-control study. Lancet 359(9324):2159-2163 (2002).
Brown, D A et al. MIC-1/GDF15 serum level and genotype: associations with progress and prognosis of colorectal carcinoma. Clin Cancer Res 9:2642-2650 (2003).
Brown, D A et al. Measurement of serum levels of macrophage inhibitory cytokine 1 combined with prostate-specific antigen improves prostate cancer diagnosis. Clin Cancer Res 12:89-96 (2006).
Brown D A et al. Serum macrophage inhibitory cytokine 1 in rheumatoid arthritis: a potential marker of erosive joint destruction. Arthritis Rheum 56(3):753-764 (2007).
De Mayer, T et al. Studying telomeres in a longitudinal population based study. Front Biosci 13:2960-2970 (2008).
Fairlie, W D et al. MIC-1 is a novel TGF-beta superfamily cytokine associated with macrophage activation. J Leukoc Biol 65(1):2-5 (1999).
Hanley, J A and B J McNeil. The meaning and use of the area under a receiver operating characteristic (ROC) curve. Radiology 143(1):29-36 (1982).
Hromas, R et al. PLAB, a novel placental bone morphogenetic protein. Biochim Biophys Acta 1354(1): 40-44 (1997).
Koopmann J et al. Serum macrophage inhibitory cytokine 1 as a marker of pancreatic and other periampultary cancers. Clin Cancer Res 10(7):2386-2392 (2004).
Koopmann J et al. Serum markers in patients with resectable pancreatic adenocarcinoma: macrophage inhibitory cytokine 1 versus CA19-9. Clin Cancer Res 12(2):442-446 (2006).
Johnen H et al. Tumor-induced anorexia and weight loss are mediated by the TGF-beta superfamily cytokine MIC-1. Nat Med 13(11):1333-1340 (2007).
Kempf, T et al. Circulating concentrations of growth-differentiation factor 15 in apparently healthy elderly individuals and patients with chronic heart failure as assessed by a new immunoradiometric sandwich assay. Clin Chem 53(2):284-291 (2007).
Lanza, E et al. The polyp prevention trial II: dietary intervention program and participant baseline dietary characteristics. Cancer Epidemiology Biomarkers Prev 5(5):385-392 (1996).
Lanza, E et al. Implementation of a 4-y, high-fiber, high-fruit-and-vegetable, low-fat dietary intervention: results of dietary changes in the Polyp Prevention Trial. Am J Clin Nutr 74(3):387-401 (2001).
Lawton, L N et al., Identification of a novel member of the TGF-beta superfamily highly expressed in human placenta. Gene 203:17-26 (1997).
Moore, A G et al. The transforming growth factor-n superfamily cytokine macrophage inhibitory cytokine-1 is present in high concentrations in the serum of pregnant women. J Clin Endocrinol Metab 85(12):4781-4788 (2000).
Morrish; D W et al. Identification by subtractive hybridization of a spectrum of novel and unexpected genes associated with in vitro differentiation of human cytotrophoblast cells. Placenta 17:431-441 (1996).
Nolan, J P and L A Sklar. Suspension array technology: evolution of the flat-array paradigm. Trends Biotechnol. 20(1):9-12 (2002).
Paralkar, V M et al. Cloning and characterization of a novel member of the transforming growth factor-beta/bone morphogenetic protein family. J Biol Chem 273 (22):13760-13767 (1998).
Selander, K S et al. Serum macrophage inhibitory cytokine-1 concentrations correlate with the presence of prostate cancer bone metastases. Cancer Epidemiology Biomarkers Prev 16(3):532-537 (2007).
Schatzkin, A et al. The polyp prevention trial I: rationale, design, recruitment, and baseline participant characteristics. Cancer Epidemiology Biomarkers Prev 5(5):375-383 (1996).
Schatzkin, A et al., Lack of effect of a low-fat, high-fiber diet on the recurrence of colorectal adenomas. Polyp Prevention. Trial Study Group. N Engl J Med 342(16):1149-1155 (2000).
Vassilev, L T et al. In vivo activation of the p53 pathway by small-molecule antagonists of MDM2. Science 303(5659):844-848 (2004).
Welsh, J B et al Analysis of gene expression identifies candidate markers and pharmacological targets in prostate cancer. Cancer Res 15(6):5974-5978 (2001).
Yokoyama-Kobayashi, M et al. Human cDNA encoding a novel TGF-beta superfamily protein highly expressed in placenta. J Biochem 122(3):622-626 (1997).
Zimmers, T A et al. Loss of GDF-15 abolishes Sulindac chemoprevention in the Apc(Min/+) mouse model of intestinal cancer. J Cancer Res Clin Oncol (2009) September 27. [Epub ahead of print].

1-16. (canceled)

17. A method of diagnosing or prognosing the presence of one or more colorectal polyps in a subject, the method comprising detecting:
(i) a change in the amount of macrophage inhibitory cytokine-1 (MIC-1);
and/or (ii) a shift in rate of change in the amount of MIC-1;
in a test body sample from said subject taken at two or more time points.

18. The method of claim 17, wherein the change in the amount of MIC-1 or shift in rate of change in the amount of MIC-1 is adjusted for the effect of at least the following factors as appropriate: the gender of the subject, the age of the subject, the body mass index (BMI) of the subject, the subject being a smoker, the subject being a user of NSAIDs, and the waist-to-hip ratio where the subject is female.

19. The method of claim 17, wherein the subject has previously had one or more polyps removed and does not have polyps present following routine assessment at a first of two or more time points, and the change in the amount of MIC-1 is a change associated with a reduced likelihood of polyp recurrence.

20. The method of claim 17, wherein the subject has previously had one or more polyps removed and is a user of non-steroidal anti-inflammatory drugs (NSAIDs), and the change in the amount of MIC-1 is associated with an increased risk of polyp recurrence.

21. The method of claim 17, wherein the test body sample is selected from the group consisting of whole blood, blood plasma, serum and urine.

22. The method of claim 17, wherein the change in the amount of MIC-1 in the test body sample is detected by comparison with a normal subject(s) by:
(i) determining the change in the amount of MIC-1 present in said test body sample; and
(ii) comparing said change against the change or a range of change in the amount of MIC-1 determined from comparative body sample(s) taken from normal subject(s) at the same or substantially equivalent time points;
such that when the degree of change in the amount of MIC-1 determined in step (i) is greater than the change or the range of change determined from the comparative body sample(s) from normal subject(s) in step (ii), there is an elevated amount of change in the amount of MIC-1 in the test body sample that is associated with the presence of one or more colorectal polyp(s).

23. The method of claim 17, wherein when a change in the amount of MIC-1, or a shift in the rate of change in the amount of MIC-1, is detected that is associated with the presence of one or more colorectal polyp(s) in the subject, the method further comprises:
(iii) treating said subject so as to remove, reduce or manage the colorectal polyp(s) that are present.

24. A method of diagnosing or prognosing a disease or condition associated with increased or over expression of macrophage inhibitory cytokine-1 (MIC-1) in a subject, the method comprising detecting:
(i) an elevated amount of MIC-1 in a test body sample from said subject, wherein the elevated amount of MIC-1 is associated with said disease or condition;
(ii) a change in the amount of MIC-1 in a test body sample from said subject taken at two or more time points, wherein the change in the amount of MIC-1 is associated with said disease or condition; and/or
(iii) a shift in rate of change in the amount of MIC-1 in a test body sample from said subject taken at two or more time points, wherein the shift in rate of change in the amount of MIC-1 is associated with said disease or condition;
wherein said elevated amount of MIC-1, change in the amount of MIC-1 or shift in rate of change in the amount of MIC-1, is adjusted for the effect of at least the following factors as appropriate: the gender of the subject, the age of the subject, the body mass index (BMI) of the subject, the subject being a smoker, the subject being a user of non-steroidal anti-inflammatory drugs (NSAIDs), and the waist-to-hip ratio where the subject is female.

25. The method of claim 24, wherein when an elevated amount of MIC-1, change in the amount of MIC-1 or shift in rate of change in the amount of MIC-1, is detected that is associated with the disease or condition in the subject, the method further comprises:
(iii) treating said subject for said disease or condition.

26. The method of claim 24, wherein the disease or condition is selected from the group consisting of cancers, cardiovascular disease, atherosclerosis and ischaemic injury, chronic inflammatory diseases, fibrotic diseases, chronic kidney disease, anorexia/cachexia and other dietary factors, miscarriage risk and/or premature birth, foetal abnormalities, oxidative stress, and environmental toxicity.

27. A method of prognosis of overall survival of an apparently healthy subject, the method comprising detecting:
(i) an elevated amount of macrophage inhibitory cytokine-1 (MIC-1) in a test body sample from said subject, wherein the elevated amount of MIC-1 is associated with an increased likelihood of death of the subject;
(ii) a change in the amount of MIC-1 in a test body sample from said subject taken at two or more time points; and/or
(iii) a shift in rate of change in the amount of MIC-1 in a test body sample from said subject taken at two or more time points;
wherein said elevated amount, change in the amount of MIC-1 or shift in rate of change in the amount of MIC-1, is adjusted for the effect of at least the following factors as appropriate: the gender of the subject, the age of the subject, the body mass index (BMI) of the subject, and the subject being a smoker.

28. The method of claim 27, wherein said elevated amount of MIC-1, change in the amount of MIC-1 or shift in rate of change in the amount of MIC-1, is further adjusted for the effect of at least the following further factors as appropriate: the subject being a user of non-steroidal anti-inflammatory drugs (NSAIDs), the waist-to-hip ratio where the subject is female.

29. The method of claim 27, wherein said elevated amount of MIC-1, change in the amount of MIC-1 or shift in rate of change in the amount of MIC-1, is further adjusted for the effect of one or more of the following further factors as appropriate: interleukin-6 (IL-6), C-reactive protein (CRP) and short telomere length in the subject.

30. The method of claim 27, when used in combination with an independent analysis of one or more other markers of mortality selected from interleukin-6 (IL-6), C-reactive protein (CRP) and short telomere length in the subject.

31. The method of claim 24, wherein the test body sample is selected from the group consisting of whole blood, blood plasma, serum and urine.

32. A method for treating or preventing colorectal polyp(s) in a subject, said method comprising administering to said subject an effective amount of an agent selected from the group consisting of macrophage inhibitory cytokine-1 (MIC-1), MIC-1 agonists and MIC-1-enhancing agents, optionally in admixture with a pharmacologically-acceptable carrier and/or excipient.
